(19) United States
(12) Patent Application Publication
(10) Pub. No.: US 2010/0036122 A1
(43) Pub. Date: Feb. 11, 2010

(54) CLEAVAGE AGENT SELECTIVELY ACTING ON SOLUBLE ASSEMBLY OF AMYLOIDOGENIC PEPTIDE OR PROTEIN

(76) Inventor: Jung Hun Suh, Seoul (KR)

Correspondence Address: BANNER & WITCOFF, LTD., 1100 13th STREET, N.W., SUITE 1200, WASHINGTON, DC, 20005-4051, US

(21) Appl. No.: 12/446,905

(22) PCT Filed: Oct. 24, 2007
(86) PCT No.: PCT/KR2007/005247

§ 371 Date: Apr. 23, 2009

(30) Foreign Application Priority Data
Oct. 24, 2006 (KR) 10-2006-0103662
Jul. 27, 2007 (KR) 10-2007-0075809

(51) Int. Cl. C07D 251/12

(52) U.S. Cl. 544/181

(57) ABSTRACT

The present invention relates to a cleavage agent and a cleavage method selectively acting on soluble assembly of amyloidogenic peptide or protein.

### TECHNICAL FIELD

The present invention relates to a cleavage agent and a cleavage method selectively acting on soluble assembly of amyloidogenic peptide or protein. The cleavage agent of the present invention inhibits biological activity of the amyloidogenic peptide or protein by cleaving the soluble assembly of amyloidogenic peptide or protein.

### BACKGROUND ART

Amyloidosis refers to a variety of conditions in which insoluble amyloid proteins are abnormally deposited in organs and/or tissues, causing disease (Bittan, G.; Fradinger, E. A.; Spring, S. M.; Teplow, D. B. Amyloid 2005, 12, 88). Various amyloidogenic peptides or proteins to generate amyloidosis are known in the art (Kelly, J. F. Curr. Opin. Struct. Biol. 1996, 6, 11). The amyloids formed from various amyloidogenic peptides or proteins maintain their intrinsic structure and function, and the amyloids have in common a fibrous form and cross beta-sheet structure.

Amyloidogenic peptides or proteins can form soluble assemblies including various oligomers and protofibrils which are converted to insoluble fibrils. According to past studies, it is believed that the soluble oligomer of amyloidogenic peptide or protein is the cause of the pathogeneses of amyloidosis (Bittan, G.; Fradinger, E. A.; Spring, S. M.; Teplow, D. B. Amyloid 2005, 12, 88). The soluble oligomer of the amyloidogenic peptide or protein, for example amyloid beta (Aβ) peptide, amylin, α-synuclein, prion, or polyglutamine, can cause Alzheimer's disease, type 2 diabetes mellitus, Parkinson's disease, spongiform encepahlopathies, or Huntington's disease (Demuro, A. G.; Mina, E.; Kayed, R.; Milton, S.; Parker, I.; Glabe, C. G. J. Biol. Chem. 2005, 280, 17294).

In the present invention, the chemical and biological properties of the soluble oligomer of amyloidogenic peptide or protein are explained by using the Alzheimer's disease as a model.

Alzheimer's disease is major cause of senile dementia. Alzheimer's disease is a degenerative brain disorder that is characterized clinically by the progressive loss of neuronal cells. Plaques consisting of Aβ peptide and neurofibrillary tangles are detected in the brains of Alzheimer's disease patients (Selkoe, D. J. Physiol. Rev. 2001, 81, 741).

The Aβ peptide is formed after sequential cleavage of the amyloid precursor protein (APP). Aβ protein is generated by successive action of β- and γ-secretases, and these secretases mainly generate oligopeptides of 40 and 42 amino acid residues in length (Sambamurti, K.; Greig, N. H.; Lahiri, D. K. Neuromol. Med. 2002, 1, 1). These oligopeptides of 40 and 42 amino acid residues in length are referred to as Aβ₄₀ or Aβ₄₂, respectively. The amino acid sequence of Aβ₄₂ is shown in FIG. 1. The amino acid sequence of Aβ₄₀ can be obtained by removing the two C-terminal amino acids from the amino acid sequence of Aβ₄₂. Aβ₄₂, the major component of amyloid plaque, is more prone to aggregation than Aβ₄₀.

The Amyloid Cascade Hypothesis was proposed in 1992 (Hardy, J. A.; Higgins, G. A. Science 1992, 256, 184). This hypothesis suggested that the mismetabolism of APP was the initiating event in AD pathogenesis, subsequently leading to the aggregation of Aβ₄₂. Formation of fibrous aggregation and neuritic plaques according to the increasement of producing Aβ₄₂ would set off further pathological events, including disruption of synaptic connections, which would lead to a reduction in neurotransmitters, and the death of tangle-bearing neurons and dementia.

Although Katzman et al have identified only a weak correlation between dementia and amyloid plaques in the Alzheimer's disease patients (Katzman, R.; Terry, R.; De Teresa, R.; Brown, T.; Davies, P.; Fuld, P.; Renbing, X.; Peck, A. Ann. Neurol. 1988, 23, 138: Naslund, J.; Haroutunian, V.; Mohs, R.; Davis, K L.; Davies, P.; Greengard, P.; Buxbaum J. D. J. Am. Med. As. 2000, 283, 1571), the Amyloid Cascade Hypothesis has been supported. Soluble Aβ₄₂ has also been detected in the brain of Alzheimer's disease patients (Kuo, Y. M.; Emmerling, M. R.; Vigo-Pelfrey, C.; Kasunic, T. C.; Kirkpatrick, J. B.; Murdoch, G. H.; Ball, M. J.; Roher, A. E. J. Biol. Chem. 1996, 271, 4077). Lue et al reported that Alzheimer's disease is related with the amount of soluble Aβ₄₂ rather than the amount of amyloid plaques (Lue, L. F.; Kuo, Y. M.; Roher, A. E.; Brachova, L.; Shen, Y.; Sue, L.; Beach, T.; Kurth, J. H.; Rydel, R. E.; Rogers, J. Am. J. Pathol. 1999, 155, 853: McLean, C. A.; Chemy, R. A.; Fraser, F. W.; Fuller, S. J.; Smith, M. J.; Beyreuther, K.; Bush, A. I.; Masters, C. L. Ann. Neurol. 1999, 46, 860). Thus, increasing attention has been turned towards soluble Aβ₄₂ and the above hypothesis has been revised.

According to the revised hypothesis (Hardy, J.; Selkoe, D. J. Science 2002, 297, 353), the reason why synaptic dysfunction occurs in the brain of Alzheimer's disease patients is not due to the insoluble amyloid fibril or Aβ monomer, but due to the soluble oligomer of Aβ. Recent studies disclosed that the soluble oligomer of Aβ₄₂, such as the dodecamer, plays a role as a neurotoxic intermediate in Alzheimer's disease (Tanzi, R. E. Nature Neurosci. 2005, 8, 977: Snyder, E. M.; Nong, Y.; Almeida, C. G.; Paul, P.; Moran, T.; Choi, E. Y.; Naim, A. C.; Salter, M. W.; Lombroso, P. J.; Gouras, G. K.; Greengard, P. Nature Neurosci. 2005, 8, 1051: Barghorn, S.; Nimmrich, V.; Striebinger, A.; Krantz, C.; Keller, P.; Janson, B.; Bahr, M.; Schmidt, M.; Bitner, R. S.; Harlan, J.; Barlow, E.; Ebert, R.; Hillen. H. J. Neurochem. 2005, 95, 834: Lesne S.; Koh, M. T.; Kotilinek, L.; Kayed, R.; Glabe, C. G.; Yang, A.; Gallagher, M.; Ashe, K. H. Nature 2006, 440, 352).

The soluble assemblies, including several oligomers or protofibrils, are formed reversibly, or partially irreversibly during the assembly process of Aβ₄₂, and then the insoluble fibril is formed irreversibly (Bitan, G.; Kirkitadze, M. D.; Lomakin, A.; Vollers, S. S.; Benedek, G. B.; Teplow, D. B. Proc. Natl. Acad. Sci. USA 2003, 100, 330: Moss, M. A.; Nichols, M. R.; Reed, D. K.; Hoh, J. H.; Rosenberry, T. L. Mol. Pharmacol. 2003, 64, 1160: Lesne S.; Koh, M. T.; Kotilinek, L.; Kayed, R.; Glabe, C. G.; Yang, A.; Gallagher, M.; Ashe, K. H. Nature 2006, 440, 352). The variable oligomers of Aβ₄₂ have their own unique structures (Urbanic, B.; Cruz, L.; Yun, S.; Buldyrev, S. V.; Bitan, G.; Teplow, D. B.; Stanley. H. E. Proc. Natl. Acad. Sci. USA 2004, 101, 17345).

A method of stimulating the removal of the oligomer of Aβ₄₂ from the brain can be a candidate for the development of a method for relieving the neurotoxicity caused by Aβ₄₂. To achieve this, Hardy et al. suggested a method which inhibits the activity of either β- or γ-secretase to prevent production of Aβ from APP (Hardy, J.; Selkoe, D. J. Science 2002, 297, 353). It is also possible to inhibit the oligomerization of Aβ by using Aβ immune agents (Schenk, D.; Barbour, R.; Dunn, W.; Gordon, G.; Grajeda, H.; Guido, T.; Hu, K.; Huang, J.; Johnson-Wood, K.; Khan, K.; Kholodenko, D.; Lee, M.; Liao, Z.; Lieberburg, I.; Motter, R.; Mutter, L.; Soriano, F.; Shopp, G.; Vasquez, N.; Vandevert, C.; Walker, S.; Wogulis, M.; Yednock, T.; Games, D.; Seubert, P. Nature 1999, 400, 173: DeMattos, R. B.; Bales, K. R.; Cummins, D. J.; Dodart, J.-C.; Paul, S. M.; Holtzman. D. M. Proc. Natl. Acad. U.S.A. 2001, 98, 8850). Aβ oligomerization can be inhibited by using small molecules which have high affinity for Aβ (Cohen, T.; Frydman-Marom, A.; Rechter, M.; Gazit, E. Biochemistry 2006, 45, 4727).

The amount of oligomer of Aβ₄₂ in the brain can be reduced by stimulating the Aβ degradation enzyme, such as endothelin converting enzyme, insulin-degrading enzyme and neprilysin (Choi, D. S.; Wang, D.; Yu, G. Q.; Zhu, G.; Kharazia, V. N.; Paredes, J. P.; Chang, W. S.; Deitchman, J. K.; Mucke, L.; Messing, R. O. Proc. Natl. Acad. Sci. 2006, 103, 8215).

As illustrated by the soluble oligomer of Aβ₄₂ related to the Alzheimer's disease, various strategies are being adopted for treating the different types of amyloidosis (Dobson, C. M. Science 2004, 304, 1259). Examples of such strategies include methods for stabilizing the amyloidogenic peptide or protein itself, inhibiting the enzyme activity which produces amyloidogenic peptide or protein from the precursor, modulating the synthesis process of amyloidogenic peptide or protein or the precursor, stimulating the elimination of the amyloidogenic peptide or protein, inhibiting the formation of fibrous plaques, preventing the accumulation of fibrous plaques precursor, and the like.

### DISCLOSURE

### Technical Problem

The present inventors have discovered a new method of cleaving the soluble assembly of the amyloidogenic peptide or protein to reduce the amount of the soluble oligomer. The synthetic cleavage molecule which selectively acts on the soluble assembly of amyloidogenic peptide or protein (hereinafter referred to as the, “cleavage agent”) is an artificial enzyme for eliminating the soluble assembly of amyloidogenic peptide or protein. The inventors of the present invention have researched to find cleavage agents that have the above properties. They have found cleavage agents by connecting the sites that selectively recognize the soluble assembly of amyloidogenic peptide or protein with the reactive portions that cleave peptide bonds. They confirmed accomplishment of the object of the present invention to reduce the amount of soluble oligomers of the amyloidogenic peptide or protein by using the cleavage agents to complete the present invention.

Accordingly, the present invention provides cleavage agents which eliminate the soluble assembly of amyloidogenic peptide or protein.

The present invention also provides a pharmaceutical composition for treatment or prevention of amyloidosis comprising the above cleavage agents and a pharmaceutically acceptable carrier.

### BRIEF DESCRIPTION OF THE DRAWINGS

FIG. 1 shows the amino acid sequence of Aβ₄₂,

FIG. 2 schematically shows the formation process for soluble and insoluble assemblies of amyloidogenic peptides or proteins,

FIG. 3 schematically shows the process of reduction of the amounts of the soluble and insoluble assemblies of amyloidogenic peptides or proteins by the cleavage agent,

FIG. 4 shows the synthesis pathway of cleavage agent A in Example 1 of the present invention,

FIG. 5 shows the fraction of Aβ₄₀ (◯) or Aβ₄₂ () (initial concentration: 4.0 μM) passing the membrane with cut-off molecular weight (MW) of 10000 after incubation at pH 7.50 and 37° C. for various periods of time (each data represents the mean value from at least 5 measurements),

FIG. 6 shows MALDI-TOF mass spectrum taken after incubation of Aβ₄₀ (4.0 μM) with cleavage agent A (3.0 μM) of Example 1 at 37° C. and pH 7.50 for 36 hours,

FIG. 7 shows MALDI-TOF mass spectrum taken after incubation of Aβ₄₂ (4.0 μM) with cleavage agent A (1.0 μM) of Example 1 at 37° C. and pH 7.50 for 36 hours,

FIG. 8 shows the plot of cleavage yield against log Cₒ/M for cleavage of Aβ₄₀ (∘) or Aβ₄₂ () (4.0 μM) by cleavage agent A of Example 1 measured after reacting for 36 hours at 37° C. and pH 7.50,

FIG. 9 shows effects of the period of preincubation of Aβ₄₀ (gray bars) or Aβ₄₂ (dark bars) (4.0 μM) on cleavage yield by cleavage agent A (3.0 μM) of Example 1 measured after reacting for 36 hours at 37° C. and pH 7.50,

FIG. 10 shows the plot of the cleavage yield against reaction time for cleavage of Aβ₄₀ (◯) or Aβ₄₂ () (4.0 μM) by cleavage agent A (3.0 μM) of Example 1 at 37° C. and pH 7.50,

FIG. 11 shows the fraction of Am (initial concentration: 4.0 μM) passing the membrane with cut-off MW of 10000 after incubation at pH 7.50 and 37° C. for various period of time,

FIG. 12 shows MALDI-TOF mass spectrum of the products purified with HPLC after incubating Am (4.0 μM) with cleavage agent A (3.2 μM) of Example 1 at 37° C. and pH 7.50 for 36 hours,

FIG. 13 shows the plot of the cleavage yield against log Cₒ/M for cleavage of Am (4.0 μM) by cleavage agent A of Example 1 measured after reacting for 36 hours at 37° C. and pH 7.50,

FIG. 14 shows the fraction of Syn (initial concentration: 70 μM) passing a 0.22 mm Millipore filter (Millipore Millex-GV 4MM) after self-assembly during incubation at pH 7.50 and 37° C. for various period of time,

FIG. 15 shows the plot of the cleavage yield against log Cₒ/M for cleavage of Syn (70 μM) by cleavage agent A of Example 1 measured after reacting for 3 days at 37° C. and pH 7.50,

FIG. 16 shows the synthesis pathway of cleavage agent B in Example 2,

FIG. 17 shows MALDI-TOF mass spectrum taken after incubation of Aβ₄₀ (4.0 μM) with cleavage agent B (3.0 μM) of Example 2 at 37° C. and pH 7.50 for 36 hours,

FIG. 18 shows MALDI-TOF mass spectrum taken after incubation of Aβ₄₂ (4.0 μM) with cleavage agent B (0.50 μM) of Example 2 at 37° C. and pH 7.50 for 36 hours.

FIG. 19 shows the plot of cleavage yield against log Cₒ/M for cleavage of Aβ₄₀ (∘) or Aβ₄₂ () (4.0 μM) by cleavage agent B of Example 2 measured after reacting for 36 hours at 37° C. and pH 7.50,

FIG. 20 shows MALDI-TOF mass spectrum of the products purified by HPLC after incubating Am (4.0 μM) with cleavage agent B (1.0 μM) of Example 2 at 37° C. and pH 7.50 for 36 hour.

FIG. 21 shows the plot of the cleavage yield against log Cₒ/M for cleavage of Am (4.0 μM) by cleavage agent B of Example 2 measured after reacting for 36 hours at 37° C. and pH 7.50,

FIG. 22 shows effects of period of preincubation of Am (4.0 μM) on cleavage yield by cleavage agent B (1.0 μM) of Example 2 measured after reacting for 36 hours at 37° C. and pH 7.50,

FIG. 23 shows the plot of the cleavage yield against reaction time for cleavage of Am (4.0 μM) by cleavage agent B (1.0 μM) of Example 2 at 37° C. and pH 7.50,

FIG. 24 shows the plot of the cleavage yield against log Cₒ/M for cleavage of Syn (70 μM) by cleavage agent B of Example 2 measured after reacting for 3 days at 37° C. and pH 7.50,

FIG. 25 shows the synthesis pathway of cleavage agent C in Example 3,

FIG. 26 shows MALDI-TOF mass spectrum taken after incubating Aβ₄₂ (4.0 μM) with cleavage agent C (1.00 μM) of Example 3 at 37° C. and pH 7.50 for 36 hours.

FIG. 27 shows the plot of cleavage yield against log Cₒ/M for cleavage of Aβ₄₀ (∘) or Aβ₄₂ () (4.0 μM) by cleavage agent C of Example 3 measured after reacting for 36 hours at 37° C. and pH 7.50,

FIG. 28 shows MALDI-TOF mass spectrum of the products purified by HPLC after incubating Am (4.0 μM) with cleavage agent C (3.2 μM) of Example 3 at 37° C. and pH 7.50 for 36 hours,

FIG. 29 shows the plot of the cleavage yield against log Cₒ/M for cleavage of Am (4.0 μM) by cleavage agent C of Example 3 measured after reacting for 36 hours at 37° C. and pH 7.50,

FIG. 30 shows the synthesis pathway of cleavage agent D in Example 4,

FIG. 31 shows MALDI-TOF mass spectrum taken after incubating Aβ₄₂ (4.0 μM) with cleavage agent D (1.00 μM) of Example 4 at 37° C. and pH 7.50 for 36 hours,

FIG. 32 shows the plot of cleavage yield against log Cₒ/M for cleavage of Aβ₄₀ (∘) or Aβ₄₂ (e) (4.0 μM) by cleavage agent D of Example 4 measured after reacting for 36 hours at 37° C. and pH 7.50,

FIG. 33 shows MALDI-TOF mass spectrum of the products purified by HPLC after incubating Am (4.0 μM) with cleavage agent D (0.38 μM) of Example 4 at 37° C. and pH 7.50 for 36 hours,

FIG. 34 shows the plot of the cleavage yield against log Cₒ/M for cleavage of Am (4.0 μM) by cleavage agent D of Example 4 measured after reacting for 36 hours at 37° C. and pH 7.50,

FIG. 35 shows the synthesis pathway of cleavage agent E in Example 5,

FIG. 36 shows MALDI-TOF mass spectrum of the products purified by HPLC after incubating Am (4.0 μM) with cleavage agent E (0.89 μM) of Example 5 at 37° C. and pH 7.50 for 36 hours,

FIG. 37 shows the plot of the cleavage yield against log Cₒ/M for cleavage of Am (4.0 μM) by cleavage agent E of Example 5 measured after reacting for 36 hours at 37° C. and pH 7.50,

FIG. 38 shows the synthesis pathway of cleavage agent F in Example 6,

FIG. 39 show MALDI-TOF mass spectrum of the products purified by HPLC after incubating Am (4.0 μM) with cleavage agent F (1.6 μM) of Example 6 at 37° C. and pH 7.50 for 36 hours,

FIG. 40 shows the plot of the cleavage yield against log Cₒ/M for cleavage of Am (4.0 μM) by cleavage agent F of Example 6 measured after reacting for 36 hours at 37° C. and pH 7.50,

FIG. 41 shows the synthesis pathway of cleavage agent G in Example 7,

FIG. 42 shows MALDI-TOF mass spectrum of the products purified by HPLC after incubating Am (4.0 μM) with cleavage agent G (0.89 μM) of Example 7 at 37° C. and pH 7.50 for 36 hours,

FIG. 43 shows the plot of the cleavage yield against log Cₒ/M for cleavage of Am (4.0 μM) by cleavage agent G of Example 7 measured after reacting for 36 hours at 37° C. and pH 7.50,

FIG. 44 shows the synthesis pathway of cleavage agent H in Example 8,

FIG. 45 shows MALDI-TOF mass spectrum of the products purified by HPLC after incubating Am (4.0 μM) with cleavage agent H (7.1 μM) of Example 8 at 37° C. and pH 7.50 for 36 hours, and

FIG. 46 shows the plot of the cleavage yield against log Cₒ/M for cleavage of Am (4.0 μM) by cleavage agent H of Example 8 measured after reacting for 36 hours at 37° C. and pH 7.50.

### TECHNICAL SOLUTION

The present invention relates to cleavage agent of formula 1 which selectively cleaves the soluble assembly of amyloidogenic peptide or protein:

(R)ₙ-(L)ₘ-Z [formula 1]

wherein,

R refers to target recognition site selected from the group consisting of A, A-(Y)ₒ—(CH₂)ₚ—(Y)ₒ-A, A-(CH═CH)-A, A-(Y)ₒ—(CH₂)ₚ—(Y)ₒ-A-(Y)ₒ—(CH₂)ₚ—(Y)ₒ-A and A-(Y)ₒ—(CH₂)ₚ—(Y)ₒ-A-(Y)ₙ—(CH₂)ₚ—(Y)ₒ-A-(O)ₒ—(CH₂)ₚ—(Y)ₒ-A,

A represents independently C₆₋₁₄aryl, or 5- to 14-membered heteroaryl having one or more hetero atom selected from the group consisting of oxygen, sulfur and nitrogen, wherein, aryl or heteroaryl is unsubstituted or substituted by one or more substituent(s) independently selected from the group consisting of C₁₋₅alkyl, hydroxy, C₁₋₁₅alkoxy, C₁₋₁₅alkylcarbonyloxy, C₁₋₅alkylsulfonyloxy, amino, mono or diC₁₋₁₅alkylamino, C₁₋₁₅alkylcarbonylamino, C₁₋₁₅alkylsulfonylamino, C₃₋₁₅cycloalkylamino, formyl, C₁₋₁₅alkylcarbonyl, carboxy, C₁₋₁₅alkyloxycarbonyl, carbamoyl, mono or diC₁₋₁₅alkylcarbamoyl, C₁₋₁₅alkylsulfanylcarbonyl, C₁₋₁₅alkylsulfanylthiocarbonyl, C₁₋₁₅alkoxycarbonyloxy, carbamoyloxy, mono or diC₁₋₁₅alkylcarbamoyloxy, C₁₋₁₅alkylsulfanylcarbonyloxy, C₁₋₁₅alkoxycarbonylamino, ureido, mono or di or triC₁₋₁₅alkylureido, C₁₋₁₅alkylsulfanylcarbonylamino, mercapto, C₁₋₁₅alkylsulfanyl, C₁₋₁₅alkyldisulfanyl, sulfo, C₁₋₁₅alkoxysulfonyl, sulfamoyl, mono or diC₁₋₁₅alkylsulfamoyl, triC₁₋₁₅alkylsilanyl and halogen;

Y is O or N-Z, wherein Z represents hydrogen or C₁₋₉alkyl;

L is linker;

Z is a metal ion-ligand complex which acts as a catalytic site;

n is independently an integer from 1 to 6;

m and o are independently 0 or 1;

p is an integer from 0 to 5.

### ADVANTAGEOUS EFFECTS

The cleavage agents according to the present invention comprise of target recognition sites which recognize the soluble assembly of amyloidogenic peptide or protein and catalytic sites which display cleavage activity, specifically cleaving peptide bonds. Thus, the cleavage agents have the capacity to recognize the soluble assembly of amyloidogenic peptide or protein and the capacity for cleaving peptide bonds. Accordingly, the cleavage agents of the present invention are effective for the selective inhibition of bioactivity of soluble oligomers of amyloidogenic peptide or protein in the presence of various kinds of biomolecules.

### [Mode of Invention]

The cleavage agents according to the present invention are specifically indicated as follows.

As explained above during the process of oligomerization and fibril formation of Aβ₄₂ as an example, various soluble oligomers and protofibrils are formed reversibly or partially irreversibly, and fibril is formed therefrom irreversibly during the assembly processes of the amyloidogenic peptide or protein (Bitan, G.; Kirkitadze, M. D.; Lomakin, A.; Vollers, S. S.; Benedek, G. B.; Teplow, D. B. Proc. Natl. Acad. Sci. USA 2003, 100, 330). Protofibril can be regarded as soluble or insoluble polymer (Moss, M. A.; Nichols, M. R.; Reed, D. K.; Hoh, J. H.; Rosenberry, T. L. Mol. Pharmacol. 2003, 64, 1160: Hull, R.; Westermark, G. T.; Westermark, P.; Kahn, S. J. Clin. Endicrinol. Metab. 204, 89, 3629). Therefore, if one or more soluble assemblies of the amyloidogenic peptide or protein are eliminated from the various soluble assemblies, the total amount of soluble assembly of amyloidogenic peptide or protein is reduced, suppressing fibril formation.

The effective cleavage agent can be obtained by mimicking the principle of enzyme's catalytic activity. In enzymatic reactions, the substrate forms a complex with the enzyme, and the enzyme converts the complexed substrate into the product. Through formation of the enzyme-substrate complex, highly effective molarity of the catalytic functional groups of the enzyme toward the substrate is attained, leading to a very high reaction rate.

The cleavage agent of formula 1 according to the present invention is an artificial enzyme.

The cleavage agent, according to the present invention, has the target recognition site which recognizes the soluble assembly of amyloidogenic peptide or protein, and thus can selectively combine with one or more soluble assembly. The target recognition site and the soluble assembly are combined, and then the catalytic site of the cleavage agent, according to the present invention, cleaves the peptide bond in the soluble assembly.

As suggested for Aβ₄₂, the generation process of various kinds of soluble assemblies and insoluble fibrils are shown as FIG. 2 (Bitan, G.; Kirkitadze, M. D.; Lomakin, A.; Vollers, S. S.; Benedek, G. B.; Teplow, D. B. Proc. Natl. Acad. Sci. USA 2003, 100, 330). In FIG. 2, the species placed in the rectangle represents soluble assemblies. The soluble assemblies include soluble oligomers and soluble protofibrils. The soluble protofibrils can be regarded as very large soluble oligomers. Conversion of large assemblies, such as protofibrils and fibrils, into smaller ones is slow, and formation of the large assemblies can be considered as irreversible or partially irreversible. For some amyloidogenic peptides or proteins, it is suggested that the fibril formation is reversible with the fibrils and the monomer being in equilibrium (Wetzel, R. Acc. Chem. Res. 2006, 39, 671). As shown in FIG. 2, reducing the concentration of one kind of assembly through cleavage can reduce the concentrations of the other assemblies which can easily be transformed into the assembly cleaved.

The cleavage agent of the present invention combines with one or more species shown in the rectangle of FIG. 2, and then cleaves the peptide bonds of the amyloidogenic peptide or protein to achieve its function. Through complex formation between the recognition site of the cleavage agent and the soluble assembly, the catalytic site of the cleavage agent is located in proximity to the peptide bonds of the amyloidogenic peptide or protein. Amyloidogenic peptide or protein is then effectively cleaved by the attack of the catalytic sites.

The reaction of cleavage agent with the target is summarized as Reaction 1 which is similar to the Michaelis-Menten equation applied to the enzyme reaction:

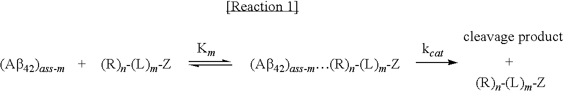

As reported for various derivatives of Co^{III} complex, if the cleavage agent acts as a catalyst and hydrolyzes the peptide bonds, the cleavage agent will be generated after cleaving the target (Jeon, J. W.; Son, S. J.; Yoo, C. E.; Hong, I. S.; Song, J. B.; Suh, J. Org. Lett. 2002, 4, 4155: Chae, P. S.; Kim, M.; Jeung, C.; Lee, S. D.; Park, H.; Lee, S.; Suh, J. J. Am. Chem. Soc. 2005, 127, 2396).

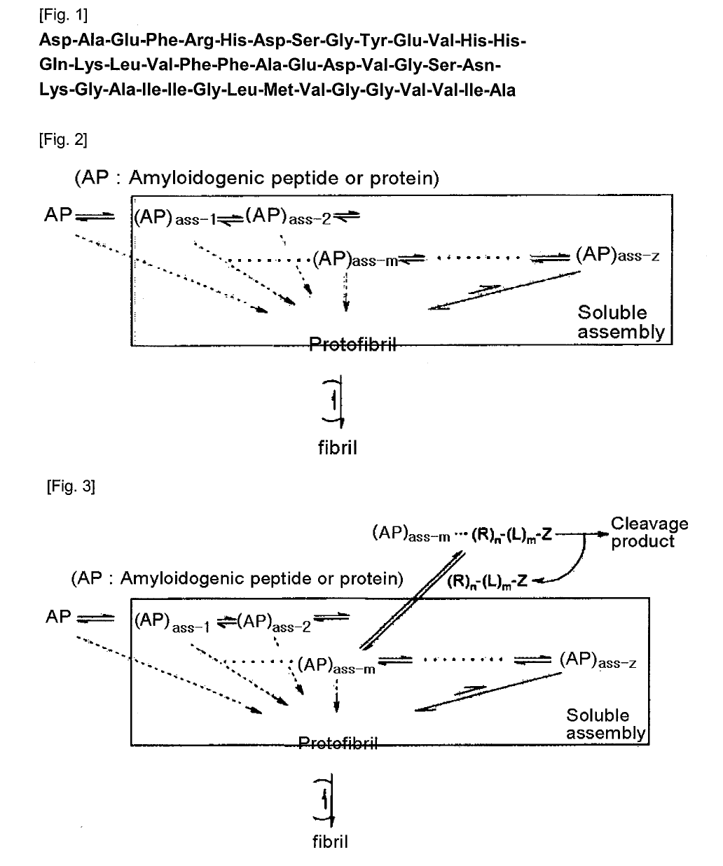

FIG. 3 (which is the combination of FIG. 2 and Reaction 1) shows the process of reducing the amounts of the soluble and insoluble assemblies of amyloidogenic peptide or protein by the action of the cleavage agent. (AP)ₐₛₛ₋ₘ in FIG. 3 represents cleaved assembly. When the concentration of the (AP)ₐₛₛ₋ₘ, which is cleaved by the cleavage agent of the present invention, is reduced, the concentrations of other assemblies which can be easily converted to (AP)ₐₛₛ₋ₘ are also reduced. However, the amounts of the assemblies including protofibrils or fibrils which cannot be easily converted to the (AP)ₐₛₛ₋ₘ are not effectively reduced. Instead, reduction of the concentration of (AP)ₐₛₛ₋ₘ slows down the formation of protofibrils or fibrils from (AP)ₐₛₛ₋ₘ.

Target Recognition Sites

To be effective, the cleavage agent needs to form a complex with the soluble assembly of amyloidogenic peptide or protein in a very low concentration of the cleavage agent. Therefore, the cleavage agent of the present invention is comprised of a target recognition site, which can selectively and strongly combine with the soluble assembly.

The interaction among aromatic side chains of amyloidogenic peptide or protein has a central role in the assembly formation (Cohen, T.; Frydman-Marom, A.; Rechter, M.; Gazit, E. Biochemistry 2006, 45, 4727). Kayed et al. reported that the soluble oligomers of various kinds of amyloidogenic peptide or protein have a common conformation-dependent structure (Kayed, R.; Head, E.; Thompson, J. L.; McIntire, T. M.; Milton, S. C.; Cotman, C. W.; Glabe, C. G. Science 2003, 300, 486).

Therefore, the organic groups having high affinity to microdomains formed by aromatic side chains can be used as the target recognition sites of the present invention without any restriction.

Preferably, the target recognition sites according to the present invention should be selected from the group consisting of A, A-(Y)ₒ—(CH₂)ₚ—(Y)ₒ-A, A-(CH═CH)-A, A-(Y)ₒ—(CH₂)ₚ—(Y)ₒ-A-(Y)ₒ—(CH₂)ₚ—(Y)ₒ-A and A-(Y)ₒ—(CH₂)ₚ—(Y)ₒ-A-(Y)ₒ—(CH₂)ₚ—(Y)ₒ-A-(O)ₒ—(CH₂)ₚ—(Y)ₒ-A.

Wherein, A represents independently C₆₋₁₄aryl, or 5- to 14-membered heteroaryl having one or more hetero atom selected from the group consisting of oxygen, sulfur and nitrogen,

Preferably, A should be selected from the group consisting of the compounds of following formulas:

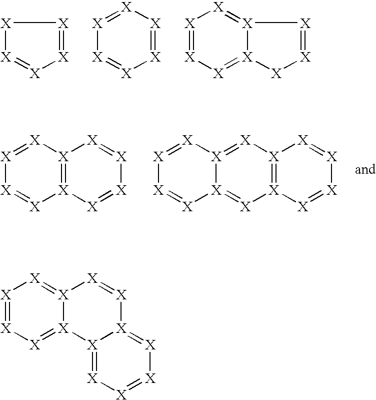

wherein,

X is independently selected from the group consisting of C, N, NH, O and S.

More preferably, A should be selected from the group consisting of the compounds of following formulas:

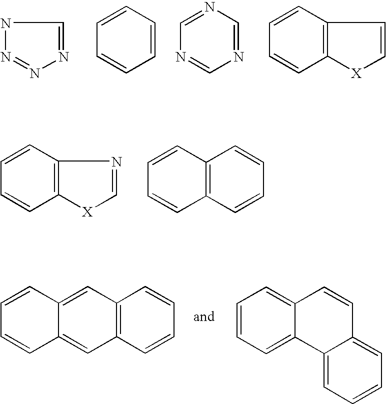

wherein,

X is NH, O, or S.

Wherein, A is unsubstituted or substituted by one or more substituent(s) independently selected from the group consisting of C₁₋₁₅alkyl, hydroxy, C₁₋₁₅alkoxy, C₁₋₁₅alkylcarbonyloxy, C₁₋₁₅alkylsulfonyloxy, amino, mono or diC₁₋₁₅alkylamino, C₁₋₁₅alkylcarbonylamino, C₁₋₁₅alkylsulfonylamino, C₃₋₁₅cycloalkylamino, formyl, C₁₋₁₅alkylcarbonyl, carboxy, C₁₋₁₅alkyloxycarbonyl, carbamoyl, mono or diC₁₋₁₅alkylcarbamoyl, C₁₋₁₅alkylsulfanylcarbonyl, C₁₋₁₅alkylsulfanylthiocarbonyl, C₁₋₁₅alkoxycarbonyloxy, carbamoyloxy, mono or diC₁₋₁₅alkylcarbamoyloxy, C₁₋₁₅alkylsulfanylcarbonyloxy, C₁₋₁₅alkoxycarbonylamino, ureido, mono or di or triC₁₋₁₅alkylureido, C₁₋₁₅alkylsulfanylcarbonylamino, mercapto, C₁₋₁₅alkylsulfanyl, C₁₋₁₅alkyldisulfanyl, sulfo, C₁₋₁₅alkoxysulfonyl, sulfamoyl, mono or diC₁₋₁₅alkylsulfamoyl, triC₁₋₁₅alkylsilanyl and halogen.

Preferably, A is unsubstituted or substituted by the substituents selected from the group consisting of C₁₋₆alkyl, C₁₋₆alkoxy, amino, mono or diC₁₋₁₂alkylamino, C₁₋₆alkylcarbonylamino, C₁₋₆alkylsulfonylamino, C₆₋₁₅cycloalkylamino, C₁₋₆alkylcarbonyl. carbamoyl, mono or diC₁₋₆alkylcarbamoyl, C₁₋₆alkoxycarbonylamino, ureido, mono or di or triC₁₋₆alkylureido, C₁₋₆alkylsulfanylcarbonylamino, C₁₋₆alkylsulfanyl, C₁₋₆alkyldisulfanyl, sulfamoyl, mono or diC₁₋₆alkylsulfamoyl, triC₁₋₆alkylsilanyl and halogen.

More preferably, A is unsubstituted or substituted by the substituents selected from the group consisting of C₁₋₄alkyl, C₁₋₄alkoxy, amino, mono or diC₁₋₈alkylamino, C₆₋₁₂cycloalkylamino, C₁₋₄alkylcarbonyl and halogen.

Wherein, Y is O or N-Z,

Z is C₁₋₉alkyl, preferably C₁₋₄alkyl.

Wherein, p is independently an integer from 0 to 5, preferably 0 to 2.

Wherein, o is independently 0 or 1.

The cleavage agent according to the present invention preferably includes 1 to 6, more preferably 1 to 4, 1 or 2 of target recognition site(s).

Catalytic Site

Several metal ions display catalytic activity in the hydrolysis of peptide bonds without the help of the organic functional groups or other metal ions (Suh, J. Acc. Chem. Res. 1992, 25, 273: Suh, J. Acc. Chem. Res. 2003, 36, 562).

The metal ion can be used as a key component in the catalytic site of the present invention based upon its catalytic activity in peptide hydrolysis.

The catalytic site of the present invention is comprised of metal ion-ligand complex. By combining the cleavage agent of the present invention with the soluble assembly of amyloidogenic peptide or protein via the target recognition site, the effective concentration between the catalytic site of the cleavage agent and the cleavage site of the target molecule is greatly increased. Therefore, the cleavage agent of the present invention can cleave the peptide bonds of the target molecules effectively.

Several metal ions that display activity for cleavage of peptides or proteins have been reported. The metal ion according to the present invention to be used in catalytic sites should be preferably selected from the group consisting of Co^{III}, Cu^{I}, Cu^{II}, Ce^{IV}, Ce^{V}, Cr^{III}, Fe^{II}, Fe^{III}, Mo^{IV}, Ni^{II}, Pd^{II}, Pt^{II}, V^{V} and Zr^{IV}, more preferably Co^{III}, Cu^{II} or Pd^{II}, and most preferably Co^{III}.

The present inventors found that in selectively cleaving soluble oligomers of amyloidogenic peptide or protein, restricting the ligand in the catalytic site to a specific structure is important in inhibiting their biological activity.

The ligand to be used in the catalytic site of the present invention is selected from the group consisting of the following compounds:

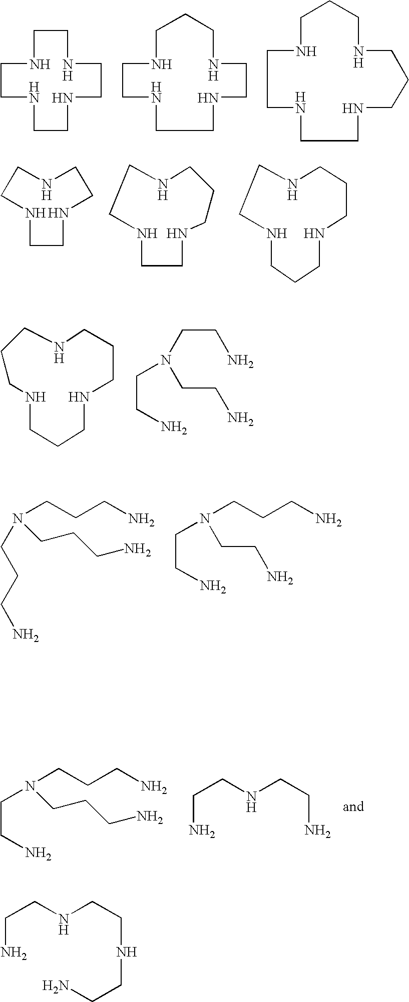

Wherein, nitrogen atom included in ligand is independently replaced with the atom selected from the group consisting of oxygen, sulfur and phosphorous;

The ligand may be fused with C₆₋₁₄aryl or 5- to 14-membered heteroaryl.

Preferably, the ligand to be used in the catalytic site is selected from the group consisting of the following formulas:

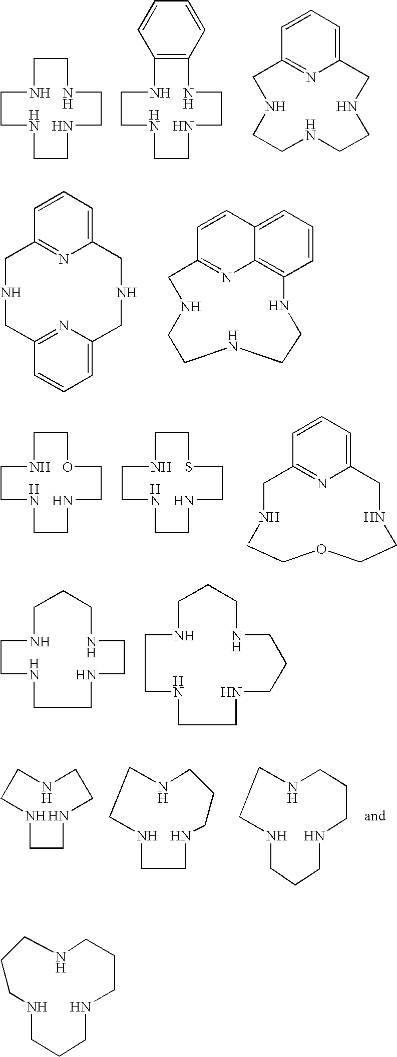

More preferably, the ligand is a cycle consisting of 12 atoms, and selected from the group consisting of the following formulas:

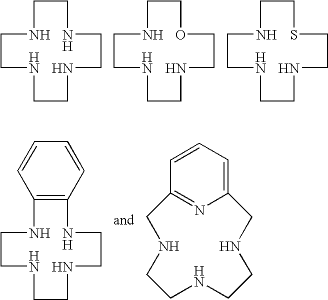

wherein, the ligand is unsubstituted or substituted with one or more substituent(s) independently selected from the group consisting of C₁₋₁₅alkyl, hydroxy, C₁₋₁₅alkoxy, C₁₋₁₅alkylcarbonyloxy, C₁₋₁₅alkylsulfonyloxy, amino, mono or diC₁₋₁₅alkylamino, C₁₋₁₅alkylcarbonylamino, C₁₋₁₅alkylsulfonylamino, formyl, C₁₋₁₅alkylcarbonyl, carboxy, C₁₋₁₅alkyloxycarbonyl, carbamoyl, mono or diC₁₋₁₅alkylcarbamoyl, C₁₋₁₅alkylsulfanylcarbonyl, C₁₋₁₅alkylsulfanylthiocarbonyl, C₁₋₁₅alkoxycarbonyloxy, carbamoyloxy, mono or diC₁₋₁₅alkylcarbamoyloxy, C₁₋₁₅alkylsulfanylcarbonyloxy, C₁₋₁₅alkoxycarbonylamino, ureido, mono or di or triC₁₋₁₅alkylureido, C₁₋₁₅alkylsulfanylcarbonylamino, mercapto, C₁₋₁₅alkylsulfanyl, C₁₋₁₅alkyldisulfanyl, sulfo, C₁₋₁₅alkoxysulfonyl, sulfamoyl, mono or diC₁₋₁₅alkylsulfamoyl, triC₁₋₁₅alkylsilanyl and halogen.

Preferably, the ligand is unsubstituted or substituted with one or more substituent(s) selected from the group consisting of C₁₋₆alkyl, C₁₋₆alkoxy, C₁₋₆alkylcarbonyloxy and halogen.

More preferably, the ligand is unsubstituted or substituted with one or more substituent(s) selected from the group consisting of C₁₋₄alkyl, C₁₋₄alkoxy and halogen.

Linker

In the cleavage agent of the present invention, the target recognition site (R) is connected through the linker, or directly to catalytic site (Z).

The modes of connection between the target recognition site and the catalytic site through the linker include the connection between one target recognition site and one catalytic site through the linker, the parallel connection of two or more target recognition sites to a catalytic site through separate linkers, the parallel connection of two or more target recognition sites to a catalytic site through a linker having a branched structure, a series connection in which two or more target recognition sites are connected to one another through a linker and one of the target recognition sites is connected to the catalytic site through a separate linker. Or, the cleavage agent can be formed by combining connection modes listed above to connect a multiple number of target recognition sites to the catalytic site:

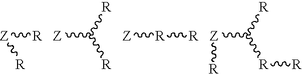

wherein, [symbol] represents linker,

R represents a target recognition site, and

Z represents a catalytic site.

The linker includes a main chain which connects the target recognition site and the catalytic site directly or connects two target recognition sites, and a substituent optionally attached to the main chain.

The target recognition site binds to the target protein, and then the catalytic site cleaves one or more of the peptide bonds in the target protein. The reactivity of the catalytic site is increased by increasing the effective concentration between the cleavage site on the protein and the catalytic site. The efficient way to modulate the effective concentration is by adjusting the relative positions between the target recognition site and the catalytic site in the cleavage agent. The length and shape of the linker can be used to modulate the relative positions.

The linker of the present invention is used to connect the target recognition site and the catalytic site. The linker of the present invention is comprised of the backbone comprising one or more atoms which is independently selected from the group consisting of carbon, nitrogen, oxygen, silicon, and phosphorous. The number of atoms included in the backbone should be between 1 and 30 but, preferably between 1 and 20, and more preferably between 1 and 15. The atoms included in the backbone of the linker are present as members of functional groups independently selected from the group consisting of alkane, alkene, alkyne, carbonyl, thiocarbonyl, amine, ether, silyl, sulfide, disulfide, sulfonyl, sulfinyl, phosphoryl, phosphinyl, amide, imide, ester and thioester.

The linker is unsubstituted or substituted with one or more substituent(s) independently selected from the group consisting of C₁₋₉alkyl, hydroxy, C₁₋₉alkoxy, C₁₋₉alkylcarbonyloxy, C₁₋₉alkylsulfonyloxy, amino, mono or diC₁₋₉alkylamino, C₁₋₉alkylcarbonylamino, C₁₋₉alkylsulfonylamino, formyl, C₁₋₉alkylcarbonyl, carboxy, C₁₋₉alkyloxycarbonyl, carbamoyl, mono or diC₁₋₉alkylcarbamoyl, C₁₋₉alkylsulfanylcarbonyl, C₁₋₉alkylsulfanylthiocarbonyl, C₁₋₉alkoxycarbonyloxy, carbamoyloxy, mono or diC₁₋₉alkylcarbamoyloxy, C₁₋₉alkylsulfanylcarbonyloxy, C₁₋₉alkoxycarbonylamino, ureido, mono or di or triC₁₋₉alkylureido, C₁₋₉alkylsulfanylcarbonylamino, mercapto, C₁₋₉alkylsulfanyl, C₁₋₉alkyldisulfanyl, sulfo, C₁₋₉alkoxysulfonyl, sulfamoyl, mono or diC₁₋₉alkylsulfamoyl, triC₁₋₉alkylsilanyl and halogen.

Preferably, the linker is unsubstituted or substituted with one or more substituent(s) independently selected from the group consisting of C₁₋₆alkyl, C₁₋₆alkoxy, mono or diC₁₋₆alkylamino, C₁₋₆alkylcarbonylamino, C₁₋₆alkylsulfonylamino, C₁₋₆alkylcarbonyl, carbamoyl, mono or diC₁₋₆alkylcarbamoyl, C₁₋₆alkoxycarbonylamino, ureido, mono or di or triC₁₋₆alkylureido, C₁₋₆alkylsulfanylcarbonylamino, C₁₋₆alkylsulfanyl, C₁₋₆alkyldisulfanyl, sulfamoyl, mono or diC₁₋₆alkylsulfamoyl, triC₁₋₆alkylsilanyl and halogen.

More preferably, the linker is unsubstituted or substituted with one or more substituent(s) independently selected from the group consisting of C₁₋₄alkyl, C₁₋₄alkoxy, mono or diC₁₋₄alkylamino, C₁₋₄alkylcarbonylamino, C₁₋₄alkylsulfonylamino, C₁₋₄alkylcarbonyl, carbamoyl, mono or diC₁₋₄alkylcarbamoyl, C₁₋₄alkoxycarbonylamino, ureido, mono or di or triC₁₋₄alkylureido, C₁₋₄alkylsulfanylcarbonylamino, C₁₋₄alkylsulfanyl, C₁₋₄alkyldisulfanyl, sulfamoyl, mono or diC₁₋₄alkylsulfamoyl, triC₁₋₄alkylsilanyl and halogen.

Persons skilled in the relevant arts should be able to design a combinatorial chemical experiment to select the linker structure suitable for modulating or changing the effective concentration between catalytic site of the synthetic catalyst and the cleavage site of the target protein.

The cleavage agent of the present invention recognizes its target via the interaction between the aromatic microdomains included in the soluble assembly of amyloidogenic peptide or protein and the aromatic component included in the target recognition site of the cleavage agent. Therefore, how many different kinds of amyloidogenic peptide or protein are used to form the soluble assembly is not important as long as the soluble assembly includes the aromatic microdomains. In other words, the soluble assembly formed by one kind of amyloidogenic peptide or protein, as well as the soluble assembly formed by two or more kinds of amyloidogenic peptide or protein can both be the target for the cleavage agent of the present invention.

Meanwhile, during the formation process of the soluble assembly, any kind of biomolecules can be incorporated into the assembly. Even when those biomolecules are present in the assembly, the soluble assembly can still be the target of the cleavage agent of the present invention.

The cleavage agent of the present invention can selectively cleave the soluble assembly of peptide or protein associated with one kind of amyloidosis, or cleave soluble assemblies of peptides or proteins associated with two or more kinds of amyloidosis.

The cleavage agent of the present invention is specifically effective for cleaving the following, but not limited to the following oligomers.

(1) Oligomers of Aβ₄₀ and Aβ₄₂ Associated with Alzheimer's Disease

Aβ₄₀ and Aβ₄₂ form various oligomers, protofibrils, and fibrils by self-assembly as shown in FIG. 2. The aggregation of Aβ₄₂ is faster than that of Aβ₄₀. Therefore, in cases where the concentration of Aβ₄₂ monomer is higher than several μM, Aβ₄₂ is oligomerized in a few minutes. It is then converted to protofibrils with sizes smaller than 0.1 μm in a solvent or on a solid surface in a few hours (Kowalewski, T.; Holtzman, D. M. Proc. Natl. Acad. Sci. USA. 1999, 96, 3688: Bitan, G.; Kirkitadze, M. D.; Lomakin, A.; Vollers, S. S.; Benedek, G. B.; Teplow, D. B. Proc. Natl. Acad. Sci. USA 2003, 100, 330). It is reported that Aβ₄₀ forms dimer, trimer, tetramer and the like in the equilibrium process, and Aβ₄₂ forms mostly pentamer and hexamer (Bitan, G.; Kirkitadze, M. D.; Lomakin, A.; Vollers, S. S.; Benedek, G. B.; Teplow, D. B. Proc. Natl. Acad. Sci. U.S.A. 2003, 100, 330-335). Recently, reports have shown that Aβ₄₂ usually exists as a mixture of its monomer and large oligomer, and Aβ₄₀ as its monomer and dimer in equilibrium (Hepler, R. W.; Grimm, K. M.; Nahas, D. D.; Breese, R.; Dodson, E. C.; Acton, P.; Keller, P. M.; Yeager, M.; Wang, H.; Shughrue, P.; Kinney, G.; Joyce, J. G. Biochemistry 2006, 45, 15157-15167).

The aggregation process of Aβ₄₀ and Aβ₄₂ is sensitive to experimental conditions (Hepler, R. W.; Grimm, K. M.; Nahas, D. D.; Breese, R.; Dodson, E. C.; Acton, P.; Keller, P. M.; Yeager, M.; Wang, H.; Shughrue, P.; Kinney, G.; Joyce, J. G. Biochemistry 2006, 45, 15157-15167).

At this stage, it is not clear what kinds of oligomers among the various kinds of oligomers formed by amyloidogenic peptide or protein are cleaved by the cleavage agent of the present invention. Nonetheless, when the concentration of the target oligomer is reduced due to cleavage by the cleavage agent, the concentrations of other oligomers which are in equilibrium with the target oligomer also decrease. Accordingly, the amount of oligomer which is the cause of amyloidosis will also be reduced.

Some cleavage agents among the cleavage agents as shown in the Examples are capable of cleaving oligomers of various kinds of amyloidogenic peptide or protein.

Cleavage agent A cleaves oligomers of Aβ₄₀ as well as those of Aβ₄₂ as in Example 1. Aβ₄₀ are mainly generated by proteolytic cleavage of the β-amyloid precursor proteins. Aβ₄₀ is responsible for various physiological functions, and therefore, if Aβ₄₀ is drastically cleaved, its normal functions would be inhibited. However, the amount of Aβ₄₀ in the brain of Alzheimer's disease patients is 30 to 40 times higher than those of nondemented elderly controls (Lue, L. F.; Kuo, Y. M.; Roher, A. E.; Brachova, L.; Shen, Y.; Sue, L.; Beach, T.; Kurth, J. H.; Rydel, R. E.; Rogers, J. Am. J. Pathol. 1999, 155, 853-862). Therefore, partial cleavage of Aβ₄₀ during cleavage of Aβ₄₂ may not cause considerably side effects in Alzheimer's disease patients.

The antibody raised against Aβ₄₂ oligomer can recognize the Aβ₄₀ oligomer as well as the Aβ₄₂ oligomer, and oligomers of other amyloidogenic proteins or peptides, such as α-synuclein, amylin, polyglutamine, lysozyme, insulin, prion peptide 106-126 (Kayed, R.; Head, E.; Thompson, J. L.; McIntire, T. M.; Milton, S. C.; Cotman, C. W.; Glabe, C. G. Science 2003, 300, 486-489). This was taken to indicate that different types of soluble amyloid oligomers have a common conformation-dependent structure, and has prompted the speculation that different types of amyloidosis may be blocked by one single drug.

Some cleavage agents in the Examples are capable of cleaving oligomers of two or more kinds of amyloidogenic peptides or proteins in agreement with the antibody study.

(2) Oligomer of Amylin Associated with Type 2 Diabetes Mellitus

The oligomer of amylin (Am; human islet amyloid polypeptide) has been reported as one of the causes of type 2 diabetes mellitus (Janson, J.; Ashley, R. H.; Harrison, D.; McIntyre, S.; Butler, P. C. Diabetes 1999, 48, 491-498: Kayed, R.; Head, E.; Thompson, J. L.; McIntire, T. M.; Milton, S. C; Cotman, C. W.; Glabe, C. G. Science 2003, 300, 486-489: Kayed, R.; Sokolov, Y.; Edmonds, B.; McIntire, T. M.; Milton, S. C.; Hall, J. E.; Glabe, C. G. J. Biol. Chem. 2004, 279, 46363-46366: Meier, J. J.; Kayed, R.; Lin, C.-Y.; Gurlo, T.; Haataja, L.; Jayasinghe, S.; Langen, R.; Glabe, C. G.; Butler, P. C. Am. J. Endicrinol. Metab. 2006, 291, E1317-E1324: Ritzel, R. A.; Meier, J. J.; Lin, C.-Y.; Veldhuis, J. D.; Butler, P. C. Diabetes 2007, 56, 65-71: Lin, C. Y.; Gurlo, T.; Kayed, R.; Butler, A. E.; Haataja, L.; Glabe, C. G.; Butler, P. C. Diabetes 2007, 56, 1324-1332). Am is a cyclic oligopeptide consisting of 37 amino acid residues, and is capable of forming amyloids by self-assembly.

As shown in the Examples, the cleavage agents of the present invention cleave the oligomer of Am. It is not clear which oligomers among the various oligomers of Am are cleaved by the cleavage agents of the present invention. However, the concentrations of the other oligomers which are equilibrium with the target decrease in accordance with the reduction of the target oligomer's concentration. Accordingly, the amount of oligomers which cause type 2 diabetes mellitus will also be reduced.

(3) Oligomer of α-synuclein Associated with Parkinson's Disease

The oligomer of α-synuclein has been reported as one of the causes of Parkinson's disease (Giasson, B. I.; Murry, I. V. J.; Trojanowski, J. Q.; Lee, V. M. J. Biol. Chem. 2001, 276, 2380-2386: Vladimir N.; Nversky, N.; Li, J.; Fink, A. L. J. Biol. Chem. 2001, 276, 10737-10744). α-synuclein (Syn) is a protein consisting of 140 amino acids and is capable of forming amyloids by self-assembly.

Pharmaceutical Compositions

The cleavage agents of the present invention cleave the soluble assembly formed by amyloidogenic peptide or protein, and inhibit the biological activity of the soluble assembly to prevent or treat amyloidosis. The present invention relates to a pharmaceutical composition for preventing or treating amyloidosis, comprising cleavage agent of formula 1 and pharmaceutically acceptable salts. Amyloidosis includes, but is not limited to, Alzheimer's disease, type 2 diabetes mellitus, Parkinson's disease, spongiform encephalopathy or Huntington's disease.

How and when the cleavage agent can be administered to a patient can be modified according to the patient's weight, sex, overall health, diet, the severity of the disease, and other drugs being taken by the patient.

The cleavage agent of the present invention can be administered by any route dictated by the targets of the cleavage agent. Accordingly, the cleavage agent of the present invention can be administered intravenously, orally, intranasally, subcutaneously, peritoneally, retroperitoneally, rectally, etc, However, the intravenous, oral, and intranasal methods are preferred.

Injection formulation, for example, sterile injection aqueous or oleaginous suspension, can be prepared through conventional methods in the art, by using suitable dispersing agents, humectants or suspension.

Water, Ringer's solution and isotonic NaCl solution can be used to prepare the above formulation, and sterile fixing oil can be used conventionally as a solvent or suspension media. Any nonirritant fixing oil including mono-, di-glyceride can be used, and fatty acids, such as oleic acid can be used in the injection formulation.

The agent of the present invention can also be formulated in oral preparation including capsules, tablets, pills, powders, granules, and the like. However, tablets and capsules are preferred, such as a enteric coated tablet or pill.

The solid administration formulations can be prepared by mixing the cleavage agent of the present invention of formula 1 with inactive diluents, such as sucrose, lactose, starch, and the like; and pharmaceutically acceptable carriers, such as, lubricants such as magnesium stearate, disintegrants, and binders.

### EXAMPLES

Having given a general description of the invention, the same will be more readily understood by reference to the following examples which are provided by way of illustration and in no way are intended to limit the present invention.

### Example 1

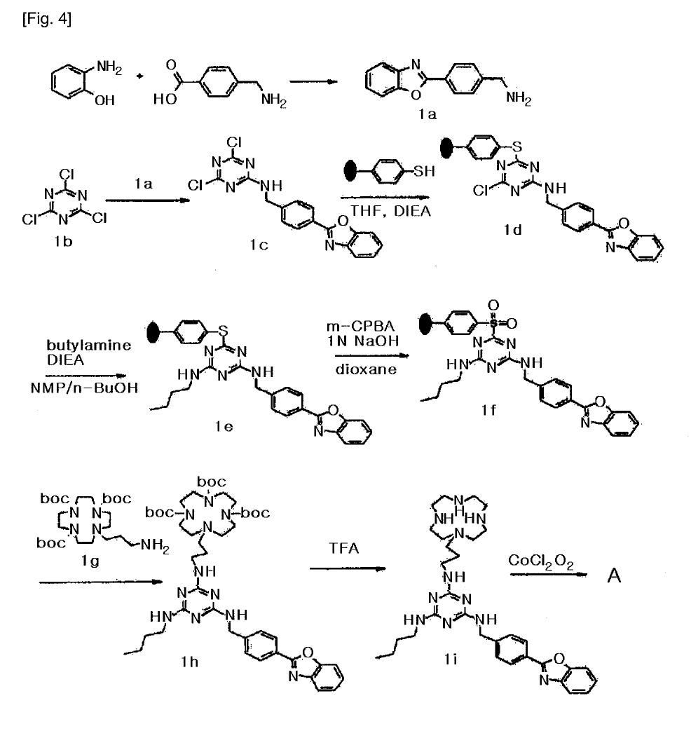

Cleavage agent A was synthesized through the pathway shown in the FIG. 4.

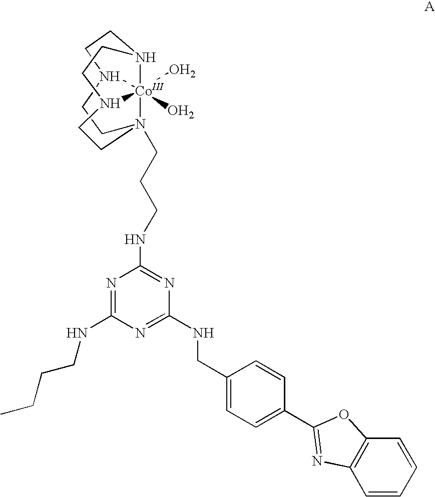

Synthesis of Compound of Formula 1a

4-Aminomethyl-benzoic acid (1.0 g, 6.8 mmol) and 2-aminophenol (0.69 g, 7.4 mmol) were mixed together with polyphosphoric acid (10 g) and heated to 170° C. under N₂ atmosphere for 1.5 hours. The reaction mixture was cooled to room temperature and poured into 10% K₂CO₃ solution. The precipitate was filtered under reduced pressure. The precipitate was recrystallized from acetone-water followed by treatment with activated charcoal in THF-water to obtain 4-benzooxazol-2-yl-benzylamine (1a).

R_{f} 0.65 (EtOAc/hexane 1:2); ¹H NMR (CDCl₃): δ 8.20 (d, 2H), 7.76 (dd, 1H), 7.66 (dd, 1H), 7.55 (d, 2H), 7.41 (m, 2H), 3.90 (s, 2H); MS (MALDI-TOF) m/z 225.33 (M+H)⁺ calcd for C₁₄H₁₃N₂O₁ 225.09.

Synthesis of Compound of Formula 1c

Cyanuric chloride (1b) (0.20 g, 1.1 mmol.), the compound of formula 1a (0.20 g 0.90 mmol), and diisopropylethylamine (DIEA) (0.38 mL, 2.7 mmol) were mixed together in THF (50 mL), and the mixture was stirred for 4 hours in an ice bath. The residue obtained by evaporation of the mixture was purified by column chromatography to obtain (4-benzooxazol-2-yl-benzyl)-(4,6-dichloro-[1,3,5]triazin-2-yl)-amine (1c).

R_{f} 0.7 (EtOAc/hexane 1:4); ¹H NMR (CDCl₃): δ 8.13 (d, 2H), 7.70 (d, 1H), 7.51 (d, 1H), 7.40 (d, 2H), 7.27 (d, 2H), 4.58 (d, 2H); ¹³C NMR (300 CDCl₃): δ 171.26, 170.17, 166.04, 162.43, 150.72, 141.96, 139.84, 128.161, 128.09, 126.96, 125.34, 124.73, 120.06, 110.65, 76.60, 45.05; MS (MALDI-TOF) m/z 372.28 (M+H)⁺, calcd for C₁₇H₁₂Cl₂N₅O 372.03.

Synthesis of Resins of Formula 1d and 1f

To a THF solution (1.5 mL) of the compound of formula 1c (55 mg, 0.15 mmol) were added PS-Thiophenol resin (purchased from Argonaut Technologies) (50 mg, 0.074 mmol) and DIEA (0.10 mL, 0.74 mmol). The mixture was heated at 65° C. and left overnight. After filtration, the resulting resin was washed with N,N-dimethylformamide (DMF), methylene chloride (MC), MeOH and MC, and dried under nitrogen gas to give a resin of formula 1d.

To the suspension of the resin of formula 1d in N-methyl-2-pyrrolidinone (NMP; 1 mL) were added n-butanol (1 mL) and butylamine (49 μL, 0.58 mmol), followed by DIEA (120 μL, 0.87 mmol). The mixture was heated at 120° C. for 8 hours. After filtration, the resin was washed with DMF, MC, MeOH and MC, and then dried under nitrogen gas to give a resin of formula 1e.

To the resin of formula 1e was added the mixture of a solution of m-chlororoperoxybenzoic acid (m-CPBA; 130 mg, 0.74 mmol) in 1,4-dioxane (1.8 mL) and aqueous 1 N NaOH (160 μL). The mixture was stirred for 4 hours at room temperature. After filtration, the resulting resin was washed with 1,4-dioxane and MC, and then dried under nitrogen gas to give a resin of formula 1f.

Synthesis of Compound of Formula 1h

To the suspension of the resin of formula 1f in acetonitrile (1.5 mL), PS-DIEA resin (purchased from Argonaut Technologies) (30 mg, 0.12 mmol) and compound of formula 1g (P. S. Chae, M. Kim, C. Jeung, S. D. Lee, H. Park, S. Lee, J. Suh, J. Am. Chem. Soc. 2005, 127, 2396-2397) (39 mg, 0.074 mmol) were added. The reaction mixture was heated at 80° C. for 8 hours. The mixture was filtered and the resin was washed with MC (1 mL×3). The combined filtrate and washing were evaporated in vacuo and the residue was purified by column chromatography to obtain 10-{3-[4-(4-benzooxazol-2-yl-benzylamino)-6-butylamino-[1,3,5]triazin-2-ylamino]-propyl}-1,4,7,10tetraaza-cyclododecane-1,4,7-tricarboxylic acid tri-tert-butyl ester (1h).

R_{f} 0.5 (EA only); ¹H NMR (CDCl₃): δ 8.13 (d, 2H), 7.70 (d, 1H), 7.51 (d, 1H), 7.40 (d, 2H), 7.27 (d, 2H), 4.58 (d, 2H), 3.8-3.2 (br, 14H), 2.5 (br, 6H), 2.1 (br, 2H), 1.63 (br, 2H), 1.5-1.3 (m, 31H), 0.9-0.8 (dd 3H); ¹³C NMR (CDCl₃): δ 164.89, 161.91, 154.33, 149.67, 142.56, 141.06, 126.72, 124.82, 124.01, 123.53, 118.89, 109.53, 78.51, 78.31, 75.58, 53.92, 52.91, 48.92, 48.08, 43.31, 39.35, 37.97, 30.86, 28.67, 27.65, 19.03, 13.10; MS (MALDI-TOF) m/z 902.99 (M+H)⁺, calcd for C₄₇H₇₂N₁₁O₇ 902.55.

The synthesis of triazine derivatives by using resin in the Examples is according to the reference (Khersonsky, S. M.; Chang, Y. T. J Comb. Chem. 2004, 6, 474) unless specifically cited in the specification.

Synthesis of Compound of Formula 1i and Cleavage Agent A

The compound of formula 1h (5 mg) was treated with 50% trifluoroacetic acid (TFA) in MC (50 μL) for 5 hours and diethyl ether (1 mL) was added to the mixture. The precipitate was separated by centrifugation, washed with diethyl ether several times, and dried under nitrogen gas to obtain the TFA salt of N-(4-benzooxazol-2-yl-benzyl)-N′-butyl-N′-[3-(1,4,7,10tetraaza-cyclododec-1-yl)-propyl]-[1,3,5]triazine-2,4,6-triamine (1i). The TFA salt of the compound of formula 1i was used for NMR and MS characterization;

¹H NMR (CDCl₃): δ 8.17 (d, 2H), 7.73 (d, 1H), 7.55 (d, 1H), 7.43 (d, 2H), 7.33 (d, 2H), 4.42 (br, 2H), 3.4-3.0 (br, 14H), 2.85 (br, 6H), 2.62 (br, 2H), 1.68 (br, 2H), 1.36-1.23 (m, 4H), 0.9-0.8 (dd 3H); ¹³C NMR (CDCl₃): δ 164.89, 162.67, 150.62, 141.76, 127.80, 126.12, 125.30, 124.73, 119.83, 110.65, 76.60, 51, 48.90, 44.87, 42.80, 42.32, 38.07, 30.89, 29.70, 23.71, 19.83, 14.07; MS (MALDI-TOF) MS (MALDI-TOF) m/z 602.73. (M+H)⁺, calcd for C₃₂H₄₈N₁₁O 602.40; HRMS m/z 602.4043. (M+H)⁺, calcd for C₃₂H₄₈N₁₁O 602.4038.

To the solution obtained by dissolving the TFA salt of the compound of formula 1i in methanol in a concentration of about 5 mg/50 μL, 5-7 equivalents of LiOH, followed by an equivalent amount of CoCl₂.H₂O were added according to the reference (Kim, M. G.; Kim, M.-s.; Lee, S. D.; Suh, J. J. Inorg. Biol. Chem. 2006, 11. 867) to prepare the corresponding Co^{II} complex. The complex was stirred for 1 day in the air to oxidize the Co^{II} complex to the Co^{III} complex.

Oxidation of Co^{II} to Co^{III} was accompanied by appearance of deep violet color. The Co^{III} complex was isolated with HPLC by detecting at 545 nm, and evaporated to produce a solid. The solid was dissolved in 0.1 M NaOH solution, and left at 37° C. for 1 hour. The solution was neutralized with HCl to pH 6-8, and left at room temperature for several days to obtain the stock solution of cleavage agent A. The cobalt content was measured by ICP to determine the concentration of the cleavage agent in the solution.

Activity Test of Cleavage Agent A

(1) Cleavage of Oligomers of Aβ₄₀ and Aβ₄₂ Associated with Alzheimer's Disease

The activity of each cleavage agent was tested at 37° C. and pH 7.50 (0.050 M phosphoric acid) in Eppendorf tubes unless indicated otherwise in the Examples.

To collect quantitative information regarding decreases in the amounts of monomer and small oligomers of Aβ₄₀ and Aβ₄₂, the following filtration experiment was conducted.

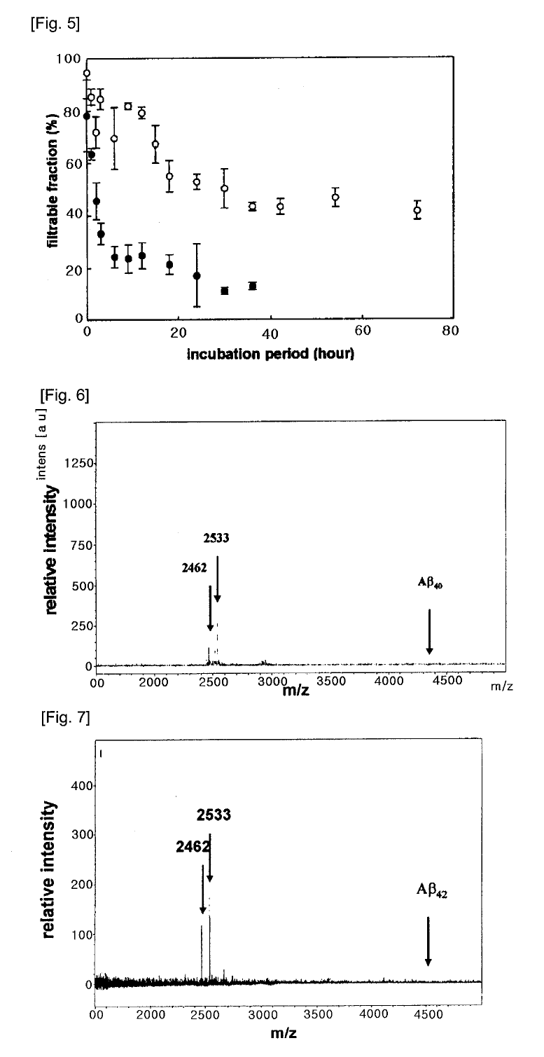

To generate the monomeric form of Aβ₄₀ or Aβ₄₂ in the early reaction stage, Aβ₄₀ or Aβ₄₂ was treated with NaOH prior to exposure to the pH 7.50 reaction medium (Fezoui, Y.; Hartley, D. M.; Harper, J. D.; Khurana, R.; Walsh, D. M.; Condron, M. M.; Selkoe, D. J.; Lansbury, P. T. Jr.; Fink, A. L.; Teplow, D. B. Amyloid 2000, 7, 166-178). The results for measurement of the fraction of Aβ₄₀ (◯) or Aβ₄₂ () (initial concentration: 4.0 μM) passing the membrane with cut-off MW of 10000 after incubation at pH 7.50 and 37° C. for various periods of self-assembly are illustrated in FIG. 5. According to the results, most (>80%) of Aβ₄₂ (MW 4514) passes the filter immediately after exposure to the pH 7.50 medium. During the filtration through the membrane which takes about 10 minutes, large oligomers which cannot pass the membrane may have been generated. Therefore, it appears that most of the Aβ₄₂ passed the membrane.

It is well known in the art that the dimer and trimer of Aβ₄₂ are produced in much lower concentrations than the monomer (Bitan, G.; Kirkitadze, M. D.; Lomakin, A.; Vollers, S. S.; Benedek, G. B.; Teplow, D. B. Proc. Natl. Acad. Sci. USA 2003, 100, 330; Hepler, R. W.; Grimm, K. M.; Nahas, D. D.; Breese, R.; Dodson, E. C.; Acton, P.; Keller, P. M.; Yeager, M.; Wang, H.; Shughrue, P.; Kinney, G.; Joyce, J. G. Biochemistry 2006, 45, 15157-15167), and thus Aβ₄₂ exists mostly as the monomer in the early reaction stage. After 3 or 36 hours, ⅔ or 90%, respectively, of Aβ₄₂ is converted to large assemblies which cannot pass the membrane. However, in case of Aβ₄₀, more than 90% of the Aβ₄₀ passed the membrane in the early reaction state, and after 24 hours, 50% of Aβ₄₀ passed the membrane.

The MALDI-TOF mass spectrum obtained by reacting Aβ₄₀ or Aβ₄₂ (4.0 μM) with cleavage agent A are illustrated in FIG. 6 or FIG. 7, respectively. As shown in these Figures, Aβ₄₀ and Aβ₄₂ are cleaved by cleavage agent A. Aβ₁₋₂₀ and Aβ₁₋₂₁ were included in the cleavage products (in the Examples herein, Aβ fragments are named according to the amino acid sequence of Aβ₄₂, and the structures of the cleavage products with the m/z values assigned to the peaks were confirmed with MALDI LIFT-TOF/TOF MS).

Since the intensity of a MALDI-TOF MS peak does not stand for the relative concentration, some oligopeptide fragments may be present in significant concentrations without showing strong MALDI-TOF MS peaks.

Unless specifically described in the Examples, cleavage reaction was initiated by adding Aβ₄₀ or Aβ₄₂ to the solution containing the cleavage agent, and the cleavage yield was estimated through the following process.

A product solution formed by the cleavage reaction was passed through the membrane with a cut-off MW of 10000 to remove aggregates of Aβ₄₀ or Aβ₄₂. Thereafter, the cleavage products were separated by HPLC, and the total amount of the cleavage products was estimated. The cleavage product was degraded to amino acids by alkaline hydrolysis, and then, the total amount of amino acids was estimated with fluorescamine to determine the total amount of the cleavage product. The cleavage yield was calculated by comparing the amount of the cleavage product with the initially added amount of the Aβ₄₀ or Aβ₄₂.

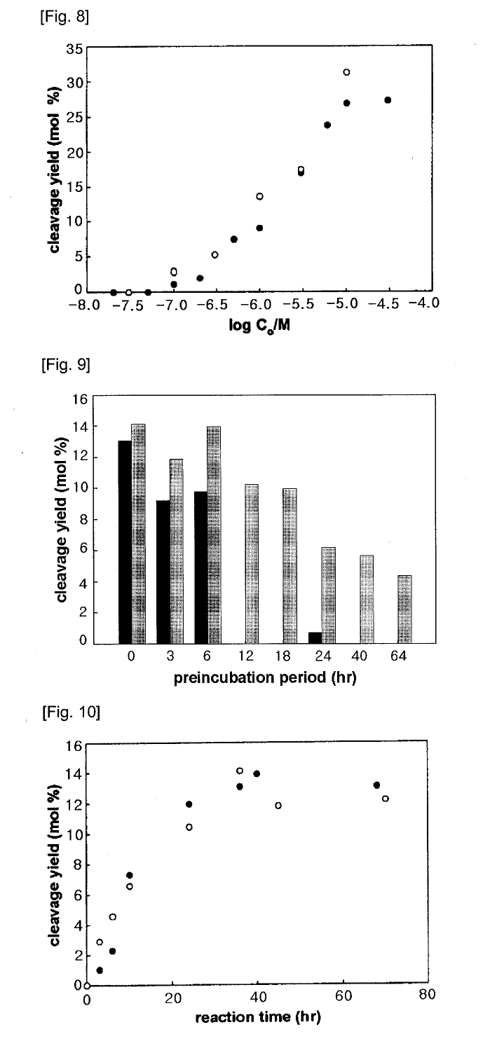

The cleavage yield measured by incubating Aβ₄₀ or Aβ₄₂ (4.0 μM) with various concentrations of cleavage agent A at pH 7.50 and 37° C. for 36 hours is illustrated in FIG. 8. The cleavage yields of Aβ₄₀ or Aβ₄₂ in the Examples are the mean value measured by using 4˜6 different reaction mixtures. The relative standard deviation (% RSD) of each cleavage yield is 5-15%.

Aβ₄₀ or Aβ₄₂ was incubated in the buffer solution for various periods of time for self-assembly before reacting with cleavage agent A. The cleavage yield was again measured and the results are summarized in FIG. 9.

To obtain information on the progress of the cleavage reaction, the cleavage yield was measured by reacting Aβ₄₀ or Aβ₄₂ with cleavage agent A for various period of time at 37° C. and pH 7.50 and the results are summarized in FIG. 10.

According to the results of FIG. 9, when cleavage agent A was added to the reaction mixture after preincubation of Aβ₄₂ for 24 hours, little cleavage was observed apparently due to polymerization of Aβ₄₂ leading to formation of protofibrils or fibrils. When cleavage agent A was added to the reaction mixture after preincubation of Aβ₄₂ for 3-6 hours, the cleavage yields were not much smaller than that obtained with cleavage agent A added initially without preincubation of Aβ₄₂. This stands in contrast with the results of FIG. 5, which show considerable reduction of the amount of Aβ₄₂ monomer during the initial 3-6 hour period. These results indicated that the oligomer is cleaved by the cleavage agent A and not the Aβ₄₂ monomer, protofibril, or fibril.

Addition of cleavage agent A after preincubation of Aβ₄₀ for 24 hours leads to considerable peptide cleavage and the preincubation for longer periods reduces the cleavage yield. This is consistent with the slower formation of protofibrils and fibrils by Aβ₄₀ compared with Aβ₄₂. In addition, it reveals that the protofibrils or fibrils of Aβ₄₀ are not cleaved by cleavage agent A. Since the yield for cleavage of Aβ₄₀ by cleavage agent A does not decrease considerably by preincubation for 3-18 hours, the monomer of Aβ₄₀ is not the main source of the fragments in view of results of the filtration experiment.

The plateau value of the cleavage yield of the cleavage agent A obtained at high concentration of the cleavage agent is about 30%. As Aβ₄₂ oligomers exist as transient intermediates, the cleavage of an Aβ₄₂ oligomer by a cleavage agent competes with the polymerization reaction of the oligomer. Since cleavage of Aβ₄₂ with a cleavage agent is first order in the concentration of the oligomer, the half-life of the target oligomer due to cleavage is not affected by the concentration of the peptide as far as the concentration of the cleavage agent is fixed.

The polymerization reaction of the oligomer is at least second-order in peptide concentration, and the half life is increased by decreasing the concentration of peptide. The total concentration of Aβ₄₂ is much lower than 1 nM in the brains of patients of Alzheimer's disease (Lue, L. F.; Kuo, Y. M.; Roher, A. E.; Brachova, L.; Shen, Y; Sue, L.; Beach, T.; Kurth, J. H.; Rydel, R. E.; Rogers, J. Am. J. Pathol. 1999, 155, 853-862).

In the Example, cleavage reaction occurred at the concentration of 100 nM of the cleavage agent when the concentration of Aβ₄₂ was 4.0 μM. Significant cleavage reaction would occur even at concentrations of the cleavage agent considerably lower than 100 nM when the concentrations of Aβ₄₂ are lowered to the in vivo level.

(2) Cleavage of Oligomers of Amylin Associated with Type 2 Diabetes Mellitus

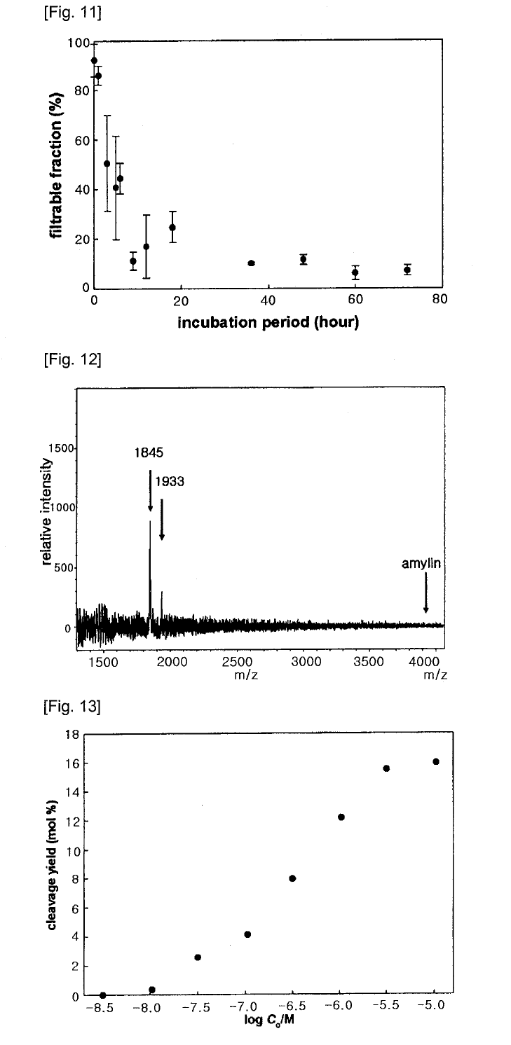

The fractions of Am passing the membrane with cut-off MW of 10000 after incubation at pH 7.50 and 37° C. for various period of time are illustrated in FIG. 11. Am monomer and small oligomers such as dimer or trimer can pass the above membrane. The amount of Am passing the filter is reduced to half or less in a few hours.

MALDI-TOF mass spectrum of the products purified with HPLC after incubating Am (4.0 μM) with cleavage agent A is illustrated in FIG. 12. In this Example and hereafter, MALDI-TOF mass spectra of cleavage products for Am were taken after purification with HPLC by the method described below.

As shown in FIG. 12, Am is cleaved by cleavage agent A. The cleavage products include Am₂₀₋₃₇ and Am₁₉₋₃₇ (the Am fragments are named according to the amino acid sequence of Am, and the structures of the cleavage products with the m/z values assigned to the peaks were confirmed with MALDI LIFT-TOF/TOF MS).

Since the intensity of a MALDI-TOF MS peak does not stand for the relative concentration, some oligopeptide fragments may be present in significant concentrations without showing strong MALDI-TOF MS peaks.

Unless specifically described in the Examples, cleavage reaction of Am was initiated by adding Am to the solution containing the cleavage agent, and the cleavage yield was estimated through the following process. The product solution obtained from the cleavage reaction was passed through the membrane (cut-off MW=10000) to remove Am aggregates, and then the cleavage product was separated by HPLC. The total amount of the cleavage product was quantified. The cleavage product was converted to amino acids by alkaline hydrolysis, and the total amount of amino acids was estimated by using fluorescamine to determine the amount of the cleavage product. The cleavage yield was calculated by comparing the amount of cleavage product with that of the initial amount of Am.

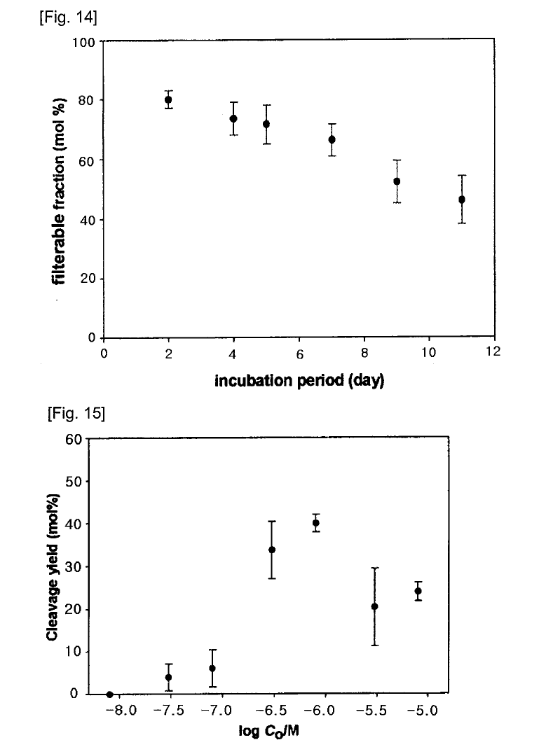

The cleavage yields measured after reacting Am (4.0 μM) with various concentrations of cleavage agent A at 37° C. and pH 7.50 for 36 hours are illustrated in FIG. 13. The cleavage yields of Am in the Examples are the mean value measured by using 4≠6 different reaction mixtures. The relative standard deviation of each cleavage yield is 5-15%.

(3) Cleavage of Oligomer of α-Synuclein Associated with Parkinson's Disease

In the Examples, the slightly modified derivatives of α-synuclein (Syn) were used as a substrate to obtain α-synuclein (Syn) by gene recombination. To facilitate purification by the nickel chelate method, histidine tag (LEHHHHHH) was adhered to C-terminus. To avoid interference in the transcription, leucine instead of methionine was incorporated as the 5^{th} amino acid residue.

To obtain information on rates for formation of large assemblies of Syn by self-assembly, the amounts of Syn passing a 0.22 mm Millipore filter after self-assembly during incubation at pH 7.50 and 37° C. for various period of time were measured and the results are summarized in FIG. 14. The results indicated that a significant amount of Syn was absorbed on the reactant container or formed protofibrils or fibrils that could not pass through the filter within a few days.

Unless the context clearly indicates otherwise, Syn cleavage was initiated by adding Syn to the solution of the cleavage agent. The cleavage yield was calculated according to the following method.

A product solution formed by the cleavage reaction was passed through the membrane with the cut-off MW of 10000 to remove Syn and its assemblies. Then, the cleavage products were separated by HPLC, and the total amount of the cleavage products was estimated. The cleavage product was degraded to amino acids through alkaline hydrolysis. The total amount of the amino acids was then estimated with fluorescamine to quantify the total amount of the cleavage product. The cleavage yield was calculated by comparing the amount of the cleavage product with the initially added amount of Syn.

The cleavage yields measured by incubating Syn (70 μM) with various concentrations of cleavage agent A at pH 7.50 and 37° C. for 3 days are illustrated in FIG. 15. The cleavage yields in the Examples are the mean value measured by using 4˜6 different reaction solutions. Since the MW of Syn used in the Examples is about 15000, some of the protein fragments formed by the cleavage of Syn might have been too large to pass through the membrane with cut-off MW of 10000. Considering this possible cause for underestimation, the cleavage yields summarized in FIG. 15 are fairly large.

(4) Reaction with Control Peptides or Proteins

The following control experiment was performed on cleavage agent A. The peptides or proteins used in the control experiment are not amyloidogenic. This control experiment was carried out to confirm that cleavage agent A did not cleave such peptides or proteins under the conditions of the Example. Two kinds of scrambled Aβ₄₂, having the same 42 amino acids as Aβ₄₂ in a scrambled sequence (KVKGLIDGAHIGDLVYEFMDSNSAIFREGVGAGHVHVAQVEF, AIAEGDSHVLKEGAYMEIFDVQGHVFGGKIFRVVDLGSHNVA) (4.0 μM), were not cleaved by incubation with cleavage agent A (3.0 μM) at pH 7.50 and 37° C. for 36 hours.

When horse heart myoglobin, bovine serum γ-globulin, bovine serum albumin, human serum albumin, egg white lysozyme, egg white ovalbumin, or bovine pancreas insulin (each 2-7 μM) was incubated with cleavage agent A (5.0 μM) at pH 7.50 and 37° C. for 36 hours, cleavage reaction was not detected.

In order to check whether the activity of cleavage agent A to cleave oligomers of Aβ₄₀, Aβ₄₂, Am, and Syn is lost when the recognition site of cleavage agent A is removed, the Co^{III} complex of cyclen (20 μM) was incubated with Aβ₄₀ (4.0 μM), Aβ₄₂ (4.0 μM), Am (4.0 μM), or Syn (70 μM) at pH 7.50 and 37° C. for 36 hours. No peptide cleavage was observed.

### Example 2

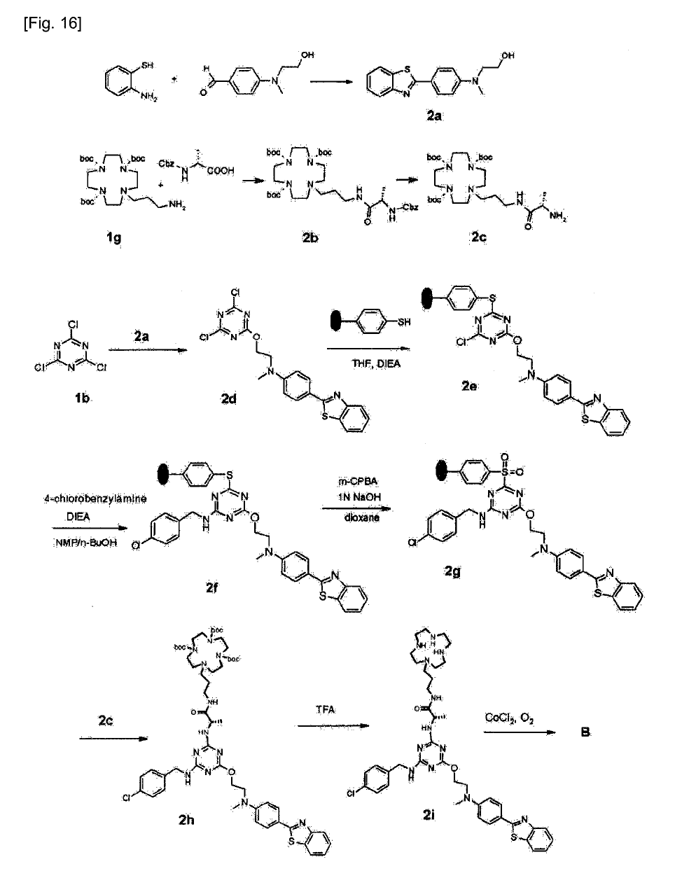

Cleavage agent B was synthesized according to the pathway shown in FIG. 16.

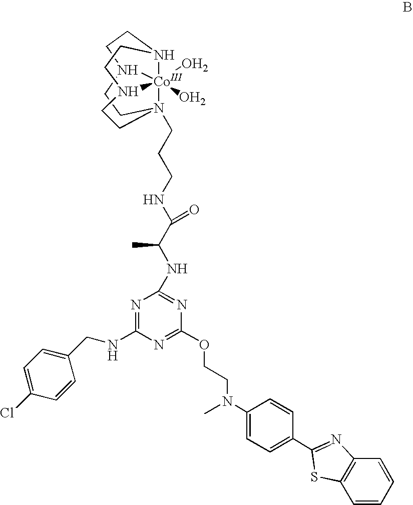

Synthesis of Compound of Formula 2a

A mixture of 2-aminothiophenol (1.3 g, 10 mmol) and N-methyl-N-(2-hydroxyethyl)-4-aminobenzaldehyde (1.8 g, 10 mmol) in dimethyl sulfoxide (10 mL) was heated to 170° C. for 1.5 hours. After cooling to room temperature, the reaction mixture was poured into water. The resulting mixture was extracted with ethyl acetate (EA) (50 mL×2). The combined organic layers were dried over Na₂SO₄. The residue obtained by evaporation of the solvent was recrystallized from acetonitrile to afford 2-[(4-benzothiazol-2-yl-phenyl)-methyl-amino]-ethanol (2a) as a yellow solid.

R_{f} 0.20 (EA/hexane 1:1); ¹H NMR (CDCl₃): δ 7.97 (d, 1H), 7.95 (d, 2H), 7.85 (d, 1H), 7.45 (t, 1H), 7.32 (t, 1H), 6.81 (d, 2H), 3.88 (t, 2H), 3.60 (t, 2H), 1.80 (br s, 3 H); ¹³C NMR (CDCl₃): δ 154.09, 151.61, 134.38, 129.03, 126.10, 124.34, 122.23, 121.67, 111.96, 77.02, 60.19, 54.66, 39.03; MS (MALDI-TOF) 285.35 m/z (M+H)⁺ calcd for C₁₆H₁₇NOS 285.10.

Synthesis of Compounds of Formulas 2b and 2c

To the stirred solution of the compound of formula 1g (2.9 g, 5.5 mmol) in acetonitrile (100 mL), N-α-Cbz-L-alanine (1.2 g, 5.5 mmol) and DIEA (2.9 mL, 17 mmol) were added. To the reaction mixture 2-(1H-benzotriazol-1-yl)-1,1,3,3-tetramethyluronium hexafluorophosphate (HBTU; 2.1 g, 5.5 mmol) was added and the mixture was stirred for 1 hour. The residue obtained by evaporation of the solution was dissolved in EA (100 mL). The EA solution was washed with 5% aq. citric acid (50 mL), 5% aq. Na₂CO₃ (50 mL), and brine (50 mL), and dried over Na₂SO₄. The solvent was evaporated off and column chromatography afforded 10-[(S)-3-(2-benzyloxycarbonylamino-propionylamino)-propyl]-1,4,7,10-tetraaza-cyclododecane-1,4,7-tricarboxylic acid tri-tert-butyl ester (2b) as a colorless oil.

R_{f} 0.2 (EA/hexane 1:1). ¹H NMR (CDCl₃): δ 7.30 (s, 5H), 5.02 (s, 2), 3.50-3.10 (br, 15H) 2.60-2.30 (br, 6H), 1.57-1.49 (br, 2H), 1.39-1.36 (m, 27H), 1.18 (s, 3H); ¹³C NMR (CDCl₃): δ 171.58, 154.82, 154.79, 154.22, 135.42, 127.55, 78.49, 65.71, 53.42, 49.56, 48.92, 47.57, 47.18, 46.53, 45.25, 37.68, 29.92, 27.64, 18.32; MS (MALDI-TOF) m/z 735.88 (M+H)⁺ calcd for C₃₇H₆₃N₆O₉ 735.46.

A suspension of the compound of formula 2b (2.0 g, 2.7 mmol) and 1.0 g of 10% Pd/C in 80 mL of EtOH was stirred under 1 atm of H₂ for 24 hours. The catalyst was filtered off on Celite, and the solvent was evaporated off to afford 10-[(S)-3-(2-benzyloxycarbonylamino-propionylamino)-propyl]-1,4,7,10-tetraaza-cyclododecane-1,4,7-tricarboxylic acid tri-tert-butyl ester (2c) as a solid.

¹H NMR (CDCl₃): δ 7.49 (s, 1H), 3.63-3.18 (br, 15H), 2.72-2.52 (br, 6H), 1.88-1.75 (br, 2H), 1.73-1.60 (m, 2H), 1.50-1.40 (m, 27H), 1.40-1.30 (d, 3H); ¹³C NMR (CDCl₃): δ 171.98, 155.03, 154.69, 154.25, 78.57, 78.42, 78.28, 53.60, 52.95, 49.72, 49.01, 47.80, 47.07, 46.54, 46.18, 37.56, 29.92, 27.64, 22.99; MS (MALDI-TOF) 601.58 m/z (M+H)⁺ calcd for C₂₉H₅₇N₆O₇ 601.42.

Synthesis of Compound of Formula 2d

The compound of formula 1b (0.20 g 1.1 mmol), the compound of formula 2a (0.20 g 0.70 mmol) and DIEA (0.38 mL, 2.7 mmol) were mixed together in THF (50 mL) and the mixture was stirred for 8 hours at room temperature. The residue obtained by evaporation of the solvent was purified by column chromatography to obtain (4-benzothiazol-2-yl-phenyl)-[2-(4,6-dichloro-[1,3,5]triazin-2-yloxy)-ethyl]-methyl-amine (2d).

R_{f} 0.7 (EA/hexane 1:4); ¹H NMR (CDCl₃): δ 7.97 (t, 3H), 7.85 (d, 1H), 7.45 (t, 1H), 7.36 (t, 1H), 6.78 (d, 2H), 4.67 (t, 2H), 3.84 (t, 2H), 3.12 (br, 3H); ¹³C NMR (CDCl₃): δ 172.53, 171.84, 168.15, 155.13, 151.39, 135.02, 129.21, 126.27, 124.59, 122.66, 122.46, 121.74, 112.14, 67.78, 50.58, 38.89; MS (MALDI-TOF) m/z 432.29 (M+H)⁺ calcd for C₁₉H₁₆Cl₂N₅OS 432.04.

Synthesis of Resins of Formula 2e and 2g

To a THF solution (1.5 mL) of the compound of formula 2d (62 mg, 0.15 mmol), PS-Thiophenol resin (purchased from Argonaut Technologies) (50 mg, 0.074 mmol) and DIEA (0.10 mL 0.74 mmol) were added. The mixture was heated at 65° C. overnight. After filtration, the resulting resin (2e) was washed with DMF, MC, MeOH, and MC (each 3 mL×3) and dried under nitrogen gas.

To a suspension of the resin of formula 2e in NMP (1 mL), n-butanol (1 mL) and 4-chlorobenzylamine (71 μL, 0.58 mmol) were added followed by DIEA (120 μL, 0.87 mmol). The mixture was heated at 120° C. for 8 hours. After filtration, the resulting resin (2f) was washed with DMF, MC, MeOH, and MC (each 3 mL×3) and dried under nitrogen gas.

The resin of formula 2f was added to the mixture of a solution of m-CPBA (130 mg, 0.74 mmol) in 1,4-dioxane (1.8 mL) and aqueous 1 N NaOH (160 μL). The mixture was shaken for 4 hours at room temperature. After filtration, the resulting resin (2g) was washed with 1,4-dioxane and MC (each 3 mL×3) and was dried under nitrogen gas.

Synthesis of Compounds of Formulas 2h and 2i and Cleavage Agent B

To the suspension of the resin of formula 2g in acetonitrile (1.5 mL), PS-DIEA resin (purchased from Argonaut Technologies) (30 mg, 0.12 mmol) and the compound of formula 2c (39 mg, 0.074 mmol) were added. The reaction tube was heated at 80° C. for 8 hours. The mixture was filtered and the resin was washed with MC (1 mL×3). The combined filtrate and washing were evaporated in vacuo and the resulting residue was purified by column chromatography to obtain 10-(3-{2-[4-{2-[(4-benzothiazol-2-yl-phenyl)-methyl-amino]-ethoxy}-6-(4-chloro-benzylamino)-[1,3,5]triazin-2-ylamino]-(S)-propionylamino}-propyl)-1,4,7,10tetraaza-cyclododecane-1,4,7-tricarboxylic acid tri-tert-butyl ester (2h).

R_{f} 0.2 (EA only); ¹H NMR (CDCl₃): δ 8.13 (d, 2H), 7.70 (d, 1H), 7.51 (d, 1H), 7.40 (d, 2H), 7.27 (d, 2H), 4.58 (d, 2H), 3.8-3.2 (br, 12H), 2.5 (br, 6H), 2.1 (br, 2H), 1.63 (br, 4H), 1.5-1.3 (m, 31H), 0.9-0.8 (dd 3H); ¹³C NMR (CDCl₃): δ 164.89, 161.91, 154.33, 149.67, 142.56, 141.06, 126.72, 124.82, 124.01, 123.53, 118.89, 109.53, 78.51, 78.31, 75.58, 53.92, 52.91, 48.92, 48.08, 43.31, 39.35, 37.97, 30.86, 28.67, 27.65, 19.03, 13.10; MS (MALDI-TOF) m/z 1102.59 (M+H)⁺, calcd for C₅₅H₇₈ClN₁₂O₈S 1102.54.

The compound of formula 2h was treated with TFA as described above in Example 1 for 1 h to obtain the TFA salt of 2-[4-{2-[(4-benzothiazol-2-yl-phenyl)-methyl-amino]-ethoxy}-6-(4-chloro-benzylamino)-[1,3,5]triazin-2-ylamino]-N-[3-(1,4,7,10-tetraaza-cyclododec-1-yl)-propyl]-(S)-propionamide (21). The TFA salt of 2i was used for NMR and MS characterization.

¹H NMR (CDCl₃): δ 7.95 (s, 1H), 7.92-7.85 (q, 3H), 7.45 (t, 1H), 7.37-7.28 (m, 5H), 6.79 (t, 2H), 4.70-4.40 (br, 2H), 3.78 (br, 2H), 3.13-2.70 (br, 15H), 2.74 (s, 3H), 2.58-2.44 (br, 6H), 1.93 (m, 1H), 1.66-1.55 (br, 2H), 1.44-1.19 (m, 5H); ¹³C NMR (CDCl₃): δ 173.57, 169.69, 168.84, 163.26, 162.01, 155.42, 152.65, 138.23, 135.63, 132.63, 130.76, 130.52, 130.07, 127.63, 125.90, 123.26, 123.08, 122.51, 113.29, 69.08, 67.48, 52.18, 51.49, 50.44, 45.22, 43.73, 43.18, 39.98, 31.51, 24.91; MS (MALDI-TOF) m/z 801.57 (M+H)⁺, calcd for C₄₀H₅₄ClN₁₂O₂S 801.39; HRMS m/z 801.3907. (M+H)⁺, calcd for C₄₀H₅₄ClN₁₂O₂S 801.3896.

The stock solution of cleavage agent B was obtained from 21 as described for cleavage agent A in Example 1.

Activity Test of Cleavage Agent B

(1) Cleavage of Oligomers of Aβ₄₀ and Aβ₄₂ Associated with Alzheimer's Disease

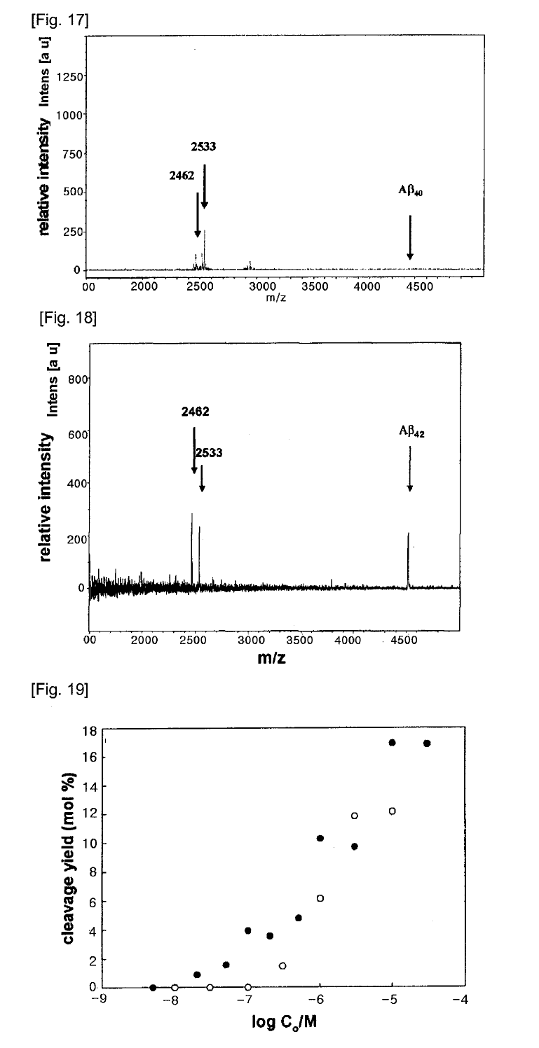

The MALDI-TOF mass spectrum obtained by reacting Aβ₄₀ or Aβ₄₂ (4.0 μM) with cleavage agent B are illustrated in FIG. 17 or FIG. 18, respectively. As shown by the Figures, Aβ₄₀ and Aβ₄₂ were cleaved by using cleavage agent B, and Aβ₁₋₂₀ and Aβ₁₋₂₁ were included in the cleavage products.

Since the intensity of a MALDI-TOF MS peak does not stand for the relative concentration, some oligopeptide fragments may be present in significant concentrations without showing strong MALDI-TOF MS peaks.

The cleavage yield measured by incubating Aβ₄₀ or Aβ₄₂ (4.0 μM) with various concentrations of cleavage agent B at pH 7.50 and 37° C. for 36 hours is illustrated in FIG. 19. The plateau value of the yield for cleavage of Aβ₄₀ and Aβ₄₂ by cleavage agent B obtained at high concentration of the cleavage agent is about 12% and 17%, respectively.

When the concentration of Aβ₄₂ was 4.0 μM, cleavage reaction was detected with 30-50 nM of cleavage agent B. If the concentration of Aβ₄₂ is lowered to the level in a living human body, significant cleavage reaction would occur even at the concentration of cleavage agent B much lower than 30-50 nM, as explained in Example 1.

(2) Cleavage of Oligomers of Amylin Associated with Type 2 Diabetes Mellitus

MALDI-TOF mass spectrum of the products purified with HPLC after incubating Am (4.0 μM) with cleavage agent B is illustrated in FIG. 20. The spectrum shows that Am was cleaved by cleavage agent B and, Am₂₀₋₃₇, Am₁₉₋₃₇ and Am₁₇₋₃₇ were included in the cleavage products.

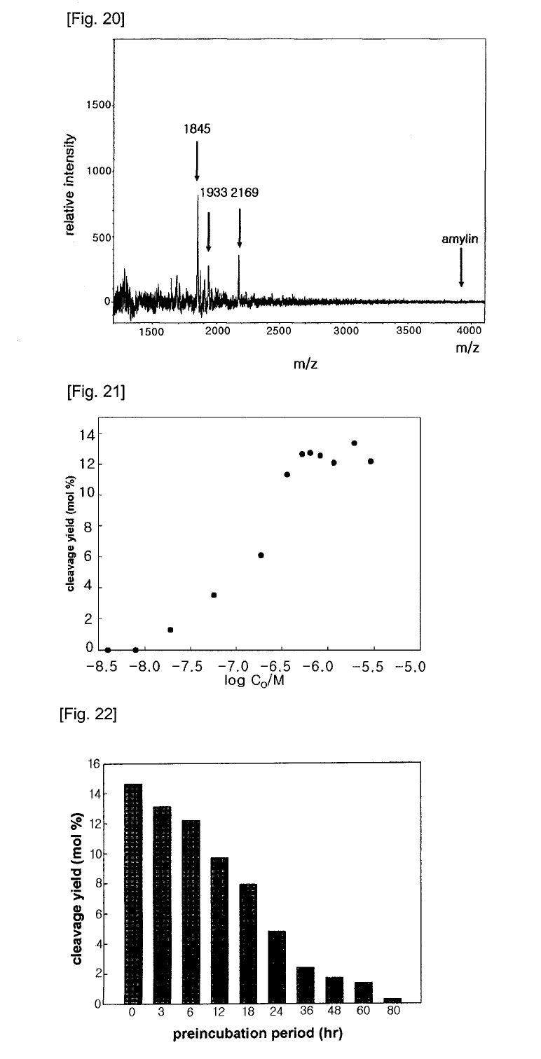

The cleavage yields measured after reacting Am (4.0 μM) with various concentrations of cleavage agent B at 37° C. and pH 7.50 for 36 hours are illustrated in FIG. 21. Cleavage yields measured after preincubation of Am (4.0 μM) over various periods before reacting with cleavage agent B (1.0 μM) are illustrated in FIG. 22.

When cleavage agent B was added to the reaction mixture after preincubation of Am for 36 hours or longer, little cleavage reaction occurred apparently due to polymerization of Am leading to formation of protofibrils or fibrils.

When cleavage agent B was added to the reaction mixture after preincubation of Am for 6 hours, the amounts of products formed by cleavage of Am were not much smaller than that obtained with B added initially without preincubation of Am. This stands in contrast with the considerable reduction of the amount of the monomer and small oligomers of Am during the initial 6 hour period shown in FIG. 11. These results indicate that the oligomers of Am instead of the monomer, protofibrils, and fibrils of Am are cleaved by cleavage agent B.

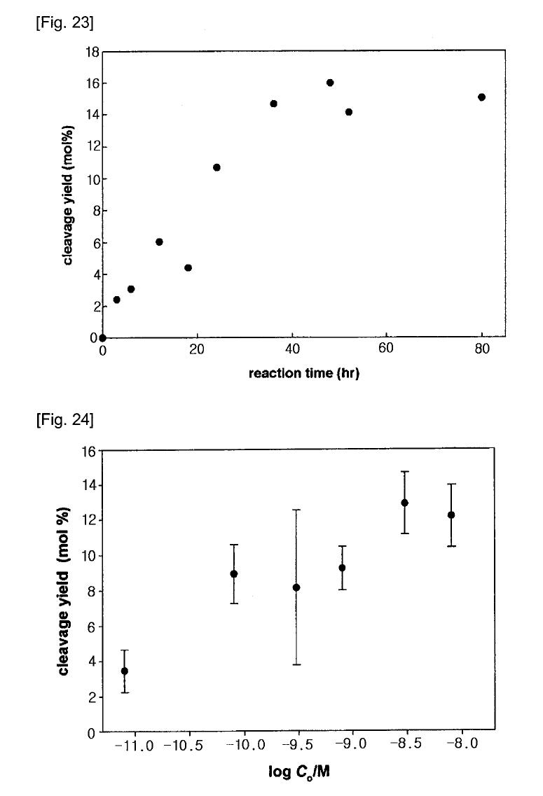

To examine the progress of the cleavage reaction, the cleavage yields measured by reacting Am with cleavage agent B for various period of time at 37° C. and pH 7.50 are summarized in FIG. 23. The results reveal that the cleavage yield does not increase even if the reaction period is increase beyond 36 hours.

(3) Cleavage of Oligomer of α-Synuclein Associated with Parkinson's Disease

The cleavage yields measured by incubating Syn (70 μM) with various concentrations of cleavage agent B at pH 7.50 and 37° C. for 3 days are illustrated in FIG. 24.

Since the MW of Syn used in the Examples is about 15000, some of the protein fragments formed by the cleavage of Syn might have been too large to pass through the membrane with cut-off MW of 10000. Considering this possible cause for underestimation, the cleavage yields summarized in FIG. 24 are fairly large.

(4) Reaction with Control Peptides or Proteins

The control experiment, identical to that of Example 1, was carried out for cleavage agent B. The results of the control experiment were the same as those obtained in Example 1.

### Example 3

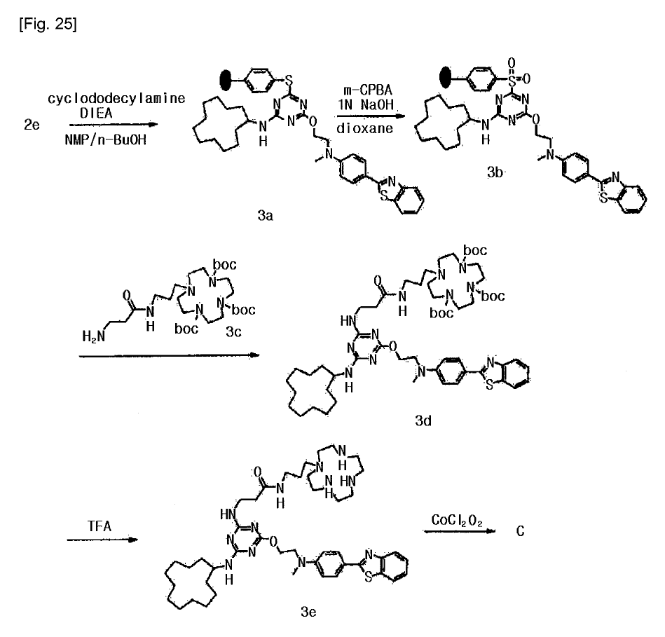

Cleavage agent C was synthesized according to the pathway shown in FIG. 25.

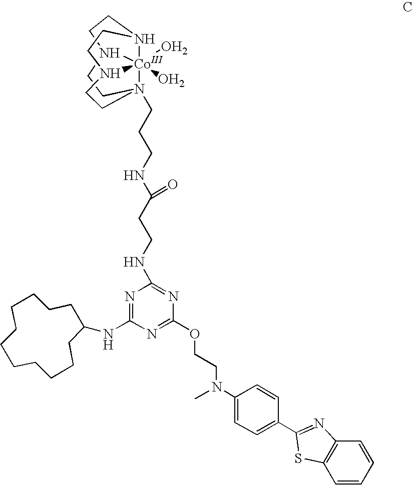

Synthesis of Resins of Formulas 3a and 3b

To a suspension of the resin of formula 2e (50 mg, 0.046 mmol) in NMP (1 mL), n-butanol (1 mL) and cyclododecylamine (66 μL, 0.51 mmol) were added followed by DIEA (63 μL, 0.36 mmol). The mixture was heated at 120° C. for 8 hours. After filtration, the resulting resin (3a) was washed with DMF, MC, MeOH, and MC (each 3 mL×3) and dried under nitrogen gas.

To the resin of formula 3a, the mixture of a solution of m-CPBA (80 mg, 0.46 mmol) in 1,4-dioxane (1.8 mL) and aqueous 1 N NaOH (93 μL) were added. The mixture was shaken for 4 hours at room temperature. After filtration, the resulting resin (3b) was washed with 1,4-dioxane and MC (each 3 mL×3) and was dried under nitrogen gas.

Synthesis of Compounds of Formulas 3d and 3e and Cleavage Agent C

To the suspension of the resin of formula 3b in acetonitrile (1.5 mL), PS-DIEA resin (purchased from Argonaut Technologies) (19 mg, 0.075 mmol) and the compound of formula 3c were added (P. S. Chae, M. Kim, C. Jeung, S. D. Lee, H. Park, S. Lee, J. Suh, J. Am. Chem. Soc. 2005, 127, 2396-2397) (28 mg, 0.046 mmol). The reaction mixture was heated at 80° C. for 8 hours. The mixture was filtered and the resin was washed with MC (1 mL×3). The combined filtrate and washing were evaporated in vacuo and the residue was purified by column chromatography to obtain 10-{3-[3-(4-{2-[(4-benzothiazol-2-yl-phenyl)-methyl-amino]-ethoxy}-6-cyclododecylamino-[1,3,5]triazin-2-ylamino)-propionylamino]-propyl}-1,4,7,10-tetraaza-cyclododecane-1,4,7-tricarboxylic acid tri-tert-butyl ester (3d).

R_{f} 0.3 (EA only); ¹H NMR (CDCl₃): δ 7.95 (t, 3H), 7.83 (d, 1H), 7.41 (t, 1H), 7.33 (br, 1H), 6.78 (m, 2H), 4.67 (br, 2H), 3.75 (br, 2H), 3.67 (br, 2H), 3.55-3.18 (br, 14H), 3.10 (s, 3H), 2.60 (br, 5H), 2.50 (br, 4H), 1.61-1.58 (br, 6H), 1.45-1.41 (m, 27H), 1.33 br, 18H); ¹³C NMR (CDCl₃): δ 184.41, 173.21, 171.70, 169.97, 168.67, 156.28, 155.60, 155.24, 154.34, 150.84, 134.47, 129.75, 128.97, 125.99, 125.01, 124.21, 122.21, 121.35, 111.57, 111.46, 79.46, 62.94, 56.52, 55.11, 51.07, 49.97, 48.36, 47.41, 46.54, 40.92, 39.27, 36.76, 35.63, 29.69, 28.62, 23.50, 21.32; MS (MALDI-TOF) m/z 1144.02 (M+H)⁺, calcd for C₆₀H₉₅N₁₂O₈S 1143.70.

The compound of formula 3d was treated with TFA as described above for the compound of 1 h in Example 1 to obtain the TFA salt of 3-(4-{2-[(4-benzothiazol-2-yl-phenyl)-methyl-amino]-ethoxy}-6-cyclododecylamino-[1,3,5]triazin-2-ylamino)-N-[3-(1,4,7,10-tetraaza-cyclododec-1-yl)-propyl]-propionamide (3e). The TFA salt of 3e was used for NMR and MS characterization

¹H NMR (CDCl₃): δ 7.94 (br, 3H), 7.83 (d, 1H), 7.45 (br, 1H), 7.35 (br, 1H), 6.75 (d, 2H), 4.57 (br, 2H), 3.81 (br, 2H), 3.63 (br, 2H), 3.3-2.9 (br, 17H), 2.76 (br, 5H), 2.44 (br, 4H), 1.65-1.61 (br, 6H), 1.31 (br, 18H); ¹³C NMR (CDCl₃): δ 188.78, 188.78, 172.02, 169.91, 169.79, 169.44, 157.24, 151.78, 133.60, 129.56, 126.89, 125.41, 125.16, 121.81, 120.57, 112.28, 112.02, 66.26, 65.57, 60.62, 50.69, 49.67, 48.68, 44.63, 42.51, 39.33, 39.16, 37.01, 30.20, 29.94, 23.82, 23.51, 21.48; MS (MALDI-TOF) m/z 843.79 (M+H)⁺, calcd for C₄₅H₇₁N₁₂O₂S 843.55; HRMS m/z 843.5547. (M+H)⁺, calcd for C₄₅H₇₁N₁₂O₂S 843.5538.

The stock solution of cleavage agent C was obtained from 3e as described for cleavage agent A in Example 1.

Activity Test of Cleavage Agent C

(1) Cleavage of Oligomers of Aβ₄₀ and Aβ₄₂ Associated with Alzheimer's Disease

When cleavage agent C (0.1-10 μM) was incubated with Aβ₄₀ (4.0 μM) at pH 7.50 and 37° C. for 36 hours, the MALDI-TOF MS data did not reveal any evidence for cleavage of Aβ₄₀.

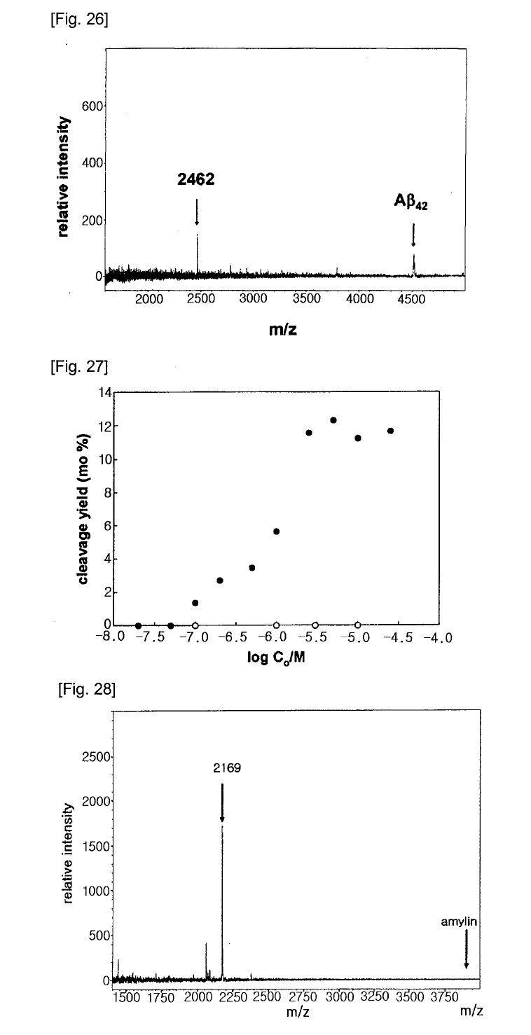

MALDI-TOF MS mass spectrum obtained by reacting Aβ₄₂ (4.0 μM) with cleavage agent C is illustrated in FIG. 26. As shown in the FIG. 26, Aβ₄₂ was cleaved by cleavage agent C and Aβ₁₋₂₀ was included in the cleavage product. Since the intensity of a MALDI-TOF MS peak does not stand for the relative concentration, some oligopeptide fragments may be present in significant concentrations without showing strong MALDI-TOF MS peaks.

The cleavage yield measured by incubating Aβ₄₀ or Aβ₄₂ (4.0 μM) with various concentrations of cleavage agent C at pH 7.50 and 37° C. for 36 hours is illustrated in FIG. 27. The plateau value of the yield for cleavage of Aβ₄₂ by cleavage agent C obtained at high concentration of the cleavage agent is about 12%. When the concentration of Aβ₄₂ was 4.0 μM, cleavage reaction was detected with 100 nM of cleavage agent C. If the concentration of Aβ₄₂ is lowered to the level in a living human body, significant cleavage reaction would occur even at concentrations of cleavage agent C much lower than 100 nM, as explained in Example 1.

(2) Cleavage of Oligomers of Amylin Associated with Type 2 Diabetes Mellitus

MALDI-TOF mass spectrum of the products purified with HPLC after incubating Am (4.0 μM) with cleavage agent C is illustrated in FIG. 28. As shown in FIG. 28, Am was cleaved by cleavage agent C, and Am₁₇₋₃₇ was included in the cleavage products.

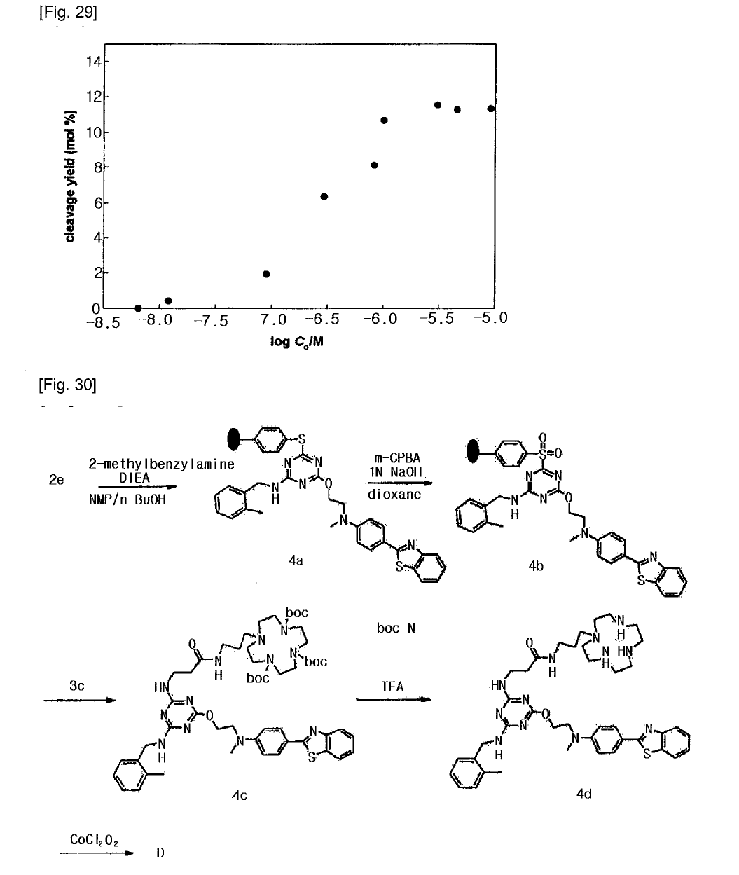

The cleavage yields measured after reacting Am (4.0 μM) with various concentrations of cleavage agent C at 37° C. and pH 7.50 for 36 hours are illustrated in FIG. 29.

(3) Reaction with Control Peptides or Proteins

The control experiment, identical to that of Example 1, was carried out for cleavage agent C. The results of the control experiment were the same as those obtained in Example 1.

### Example 4

Cleavage agent D was synthesized according to the pathway shown in FIG. 30.

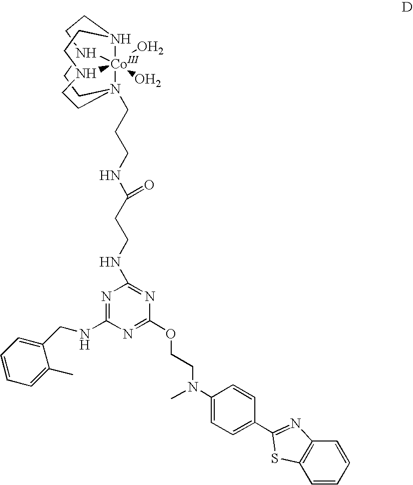

Synthesis of Resins of Formulas 4a and 4b

To a suspension of the resin of formula 2e (50 mg, 0.046 mmol) in NMP (1 mL), n-butanol (1 mL) and 2-methylbenzylamine (63 μL, 0.51 mmol) were added followed by DIEA (63 μL, 0.36 mmol). The mixture was heated at 120° C. for 8 hours. After filtration, the resulting resin (4a) was washed with DMF, MC, MeOH, and MC (each 3 mL×3) and dried under nitrogen gas.

To the resin of formula 4a, the mixture of a solution of m-CPBA (80 mg, 0.46 mmol) in 1,4-dioxane (1.8 mL) and aqueous 1 N NaOH (93 μL) were added. The mixture was shaken for 4 hours at room temperature. After filtration, the resulting resin (4b) was washed with 1,4-dioxane and MC (each 3 mL×3) and was dried under nitrogen gas.

Synthesis of Compounds of Formulas 4c and 4d and Cleavage Agent D

To the suspension of the resin of formula 4b in acetonitrile (1.5 mL), PS-DIEA resin (purchased from Argonaut Technologies) (19 mg, 0.075 mmol) and the compound of formula 3c (28 mg, 0.046 mmol) were added. The reaction mixture was heated at 80° C. for 8 hours. The mixture was filtered and the resin was washed with MC (1 mL×3). The combined filtrate and washing were evaporated in vacuo and the residue was purified by column chromatography to obtain 10-(3-{3-[4-{2-[(4-benzothiazol-2-yl-phenyl)-methyl-amino]-ethoxy}-6-(2-methyl-benzylamino)-[1,3,5]triazin-2-ylamino]-propionylamino}-propyl)-1,4,7,10-tetraaza-cyclododecane-1,4,7-tricarboxylic acid tri-tert-butyl ester (4c).

R_{f} 0.4 (EA only); ¹H NMR (CDCl₃): δ 7.96 (t, 3H), 7.83 (d, 1H), 7.43 (t, 1H), 7.32 (br, 1H), 7.15 (br, 4H), 6.77 (d, 2H), 4.53 (br, 4H), 3.75 (br, 2H), 3.66 (br, 2H), 3.5-3.1 (br, 14H), 3.09 (s, 3H), 2.58 (br, 4H), 2.49 (br, 4H), 2.32 (s, 3H), 1.60 (br, 2H), 1.46-1.41 (m, 27H); ¹³C NMR (CDCl₃): δ 186.87, 179.99, 173.28, 171.18, 168.68, 155.26, 154.35, 150.86, 136.88, 136.10, 134.50, 130.27, 128.99, 127.27, 126.09, 126.01, 125.02, 124.24, 122.24, 121.45, 121.38, 111.55, 80.31, 79.51, 63.54, 56.56, 55.10, 54.95, 50.98, 49.95, 48.35, 47.65, 40.01, 39.03, 36.93, 29.71, 28.65, 19.10; MS (MALDI-TOF) m/z 1081.89 (M+H)⁺, calcd for C₅₆H₈₁N₁₂O₈S 1081.59.

Compound of formula 4c was treated with TFA as described above in Example 1 for 1 h to obtain the TFA salt of 3-[4-{2-[(4-benzothiazol-2-yl-phenyl)-methyl-amino]-ethoxy}-6-(2-methyl-benzylamino)-[1,3,5]triazin-2-ylamino]-N-[3-(1,4,7,10-tetraaza-cyclododec-1-yl)-propyl]-propionamide (4d). The TFA salt of 4d was used for NMR and MS characterization.

¹H NMR (CDCl₃): δ 7.96 (t, 3H), 7.85 (d, 1H), 7.49 (br, 1H), 7.37 (br, 1H), 7.15 (br, 4H), 6.80 (d, 2H), 4.56 (m, 4H), 3.58 (br, 2H), 3.3-2.8 (br, 17H), 2.33 (br, 8H), 2.31 (s, 3H), 1.48 (br, 2H); ¹³C NMR (CDCl₃): δ 186.11, 180.06, 171.65, 169.85, 169.01, 151.51, 151.17, 136.08, 134.83, 132.77, 130.66, 129.65, 129.42, 127.99, 127.93, 127.82, 126.31, 126.13, 125.33, 121.76, 121.05, 112.09, 111.90, 66.12, 62.87, 53.89, 50.44, 49.72, 44.38, 44.22, 42.34, 42.09, 39.36, 36.63, 29.71, 19.07; MS (MALDI-TOF) m/z 781.73 (M+H)⁺, calcd for C₄₁H₅₇N₁₂O₂S 781.44; HRMS m/z 781.4457. (M+H)⁺, calcd for C₄₁H₅₇N₁₂O₂S 781.4443.

The stock solution of cleavage agent D was obtained from the compound of formula 4d as described for cleavage agent A in Example 1.

Activity test of Cleavage Agent D

(1) Cleavage of Oligomers of Aβ₄₀ and Aβ₄₂ Associated with Alzheimer's Disease

When cleavage agent D (0.1-10 μM) was incubated with Aβ₄₀ (4.0 μM) at pH 7.50 and 37° C. for 36 hours, the MALDI-TOF MS data did not reveal any evidence of cleavage of Aβ₄₀.

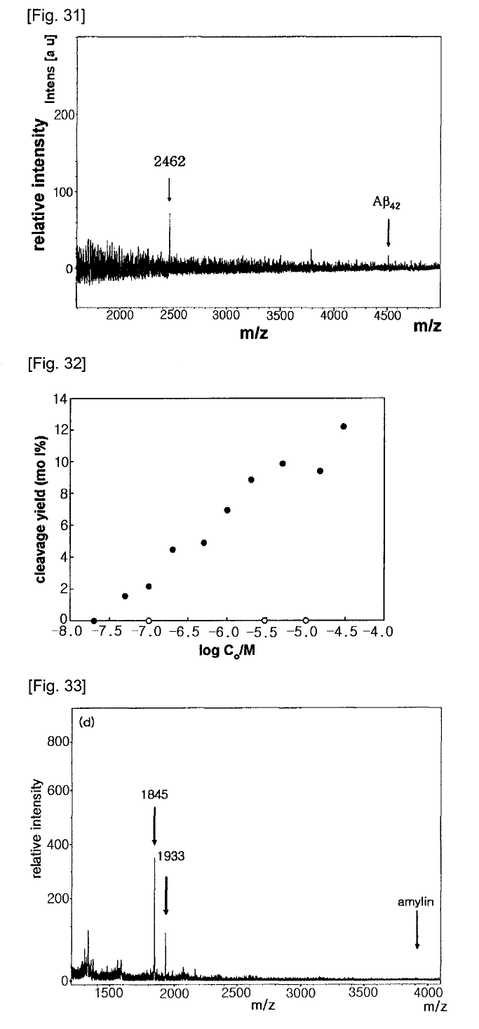

MALDI-TOF MS mass spectrum obtained by reacting Aβ₄₂ (4.0 μM) with cleavage agent D is illustrated in FIG. 31. As shown in the FIG. 31, Aβ₄₂ was cleaved by cleavage agent D and Aβ₁₋₂₀ was included in the cleavage product. Since the intensity of a MALDI-TOF MS peak does not stand for the relative concentration, some oligopeptide fragments may be present in significant concentrations without showing strong MALDI-TOF MS peaks.

The cleavage yield measured by incubating Aβ₄₀ or Aβ₄₂ (4.0 μM) with various concentrations of cleavage agent D at pH 7.50 and 37° C. for 36 hours is illustrated in FIG. 32. The plateau value of the yield for cleavage of Aβ₄₂ by cleavage agent D obtained at high concentration of the cleavage agent is about 12%. When the concentration of Aβ₄₂ was 4.0 μM, cleavage reaction was detected with 50-100 nM of cleavage agent D. If the concentration of Aβ₄₂ is lowered to the level in a living human body, significant cleavage reaction would occur even at concentrations of cleavage agent D much lower than 50-100 nM, as explained in Example 1.

(2) Cleavage of Oligomers of Amylin Associated with Type 2 Diabetes Mellitus

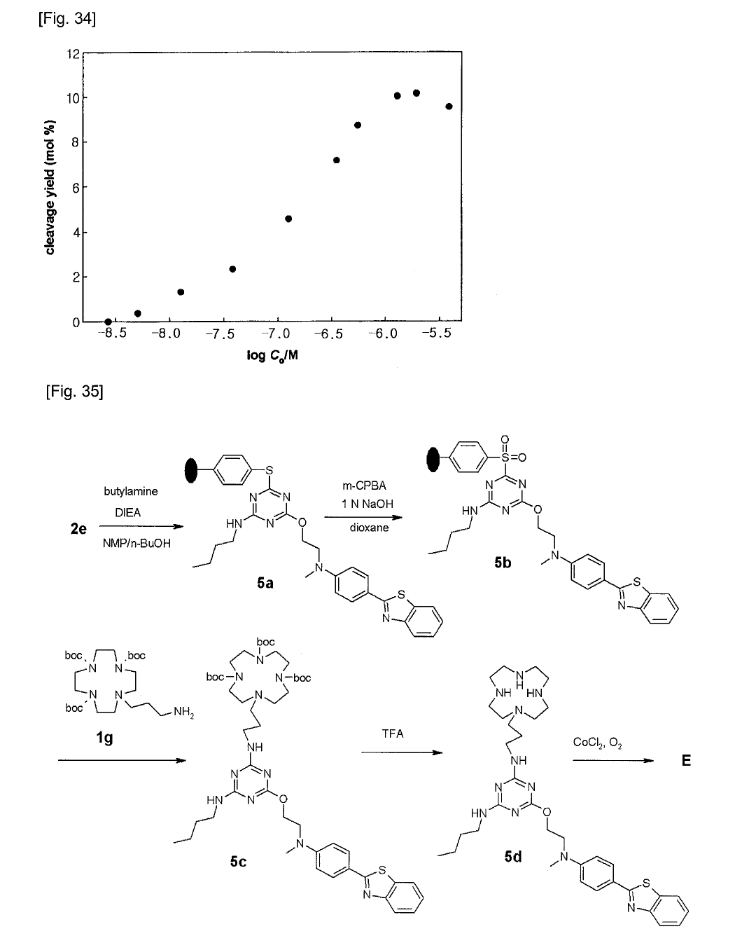

MALDI-TOF mass spectrum of the products purified with HPLC after incubating Am (4.0 μM) with cleavage agent D is illustrated in FIG. 33. As shown in FIG. 33, Am was cleaved by cleavage agent D, and Am₂₀₋₃₇ and Am₁₉₋₃₇ were included in the cleavage products.

The cleavage yields measured after reacting Am (4.0 μM) with various concentrations of cleavage agent D at 37° C. and pH 7.50 for 36 hours are illustrated in FIG. 34.

(3) Reaction with Control Peptides or Proteins

The control experiment, identical to that of Example 1, was carried out for cleavage agent D. The results of the control experiment were the same as those obtained in Example 1.

### Example 5

Cleavage agent E was synthesized according to the pathway shown in FIG. 35.

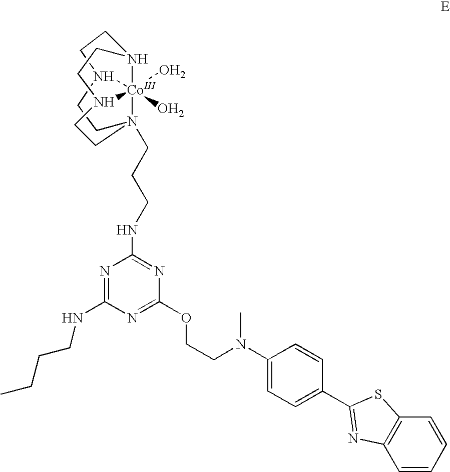

Synthesis of Resins of Formulas 5a and 5b

To the suspension of the resin of formula 2e (55 mg, 0.15 mmol) in NMP (1 mL), n-butanol (1 mL) and butylamine (49 μL, 0.58 mmol) were added followed by DIEA (120 μL, 0.87 mmol). The mixture was heated at 120° C. for 8 hours. After filtration, the resulting resin (5a) was washed with DMF, MC, MeOH, and MC (each 3 mL×3) and dried under nitrogen gas.

To the resin of formula 5a, the mixture of a solution of m-CPBA (130 mg, 0.74 mmol) in 1,4-dioxane (1.8 mL) and aqueous 1 N NaOH (160 μL) were added. The mixture was shaken for 4 hours at room temperature. After filtration, the resulting resin (5b) was washed with 1,4-dioxane and MC (each 3 mL×3) and was dried under nitrogen gas.

Synthesis of Compounds of Formulas 5c and 5d and Cleavage Agent E

To the suspension of the resin of formula 5b in acetonitrile (1.5 mL), PS-DIEA resin (30 mg, 0.12 mmol) and the compound of formula 1g (39 mg, 0.074 mmol) were added. The mixture was heated at 80° C. for 8 hours. The mixture was filtered and the resin was washed with MC (1 mL×3). The combined filtrate and washing were evaporated in vacuo and the residue was purified by column chromatography to obtain 10-[3-(4-{2-[(4-benzothiazol-2-yl-phenyl)-methyl-amino]-ethoxy}-6-butylamino-[1,3,5]triazin-2-ylamino)-propyl]-1,4,7,10-tetraaza-cyclododecane-1,4,7-tricarboxylic acid tri-tert-butyl ester (5c).

R_{f} 0.2 (EA/hexane 1:1); ¹H NMR (CDCl₃): [symbol] 7.99 (t, 2H), 7.86 (d, 1H), 7.44 (t, 1H), 7.31 (t, 1H), 6.79 (d, 2H), 4.48 (t, 2H), 3.80 (t, 2H), 3.54-3.11 (br, 16H), 3.11 (s, 3H), 2.61 (br, 6H), 1.73 (m, 2H), 1.53 (m, 2H), 1.46-1.38 (br, 27H), 1.29 (m, 2H), 0.90 (t, 3H); ¹³C NMR (CDCl₃): [symbol] 170.47, 168.58, 167.25, 156.09, 155.69, 155.35, 154.43, 154.40, 150.92, 134.53, 128.97, 125.97, 124.19, 122.29, 121.56, 121.33, 111.57, 79.55, 79.34, 76.61, 62.54, 55.04, 54.16, 51.13, 50.02, 47.97, 40.52, 39.09, 31.75, 29.68, 28.66, 28.50, 24.93, 20.01, 13.79; MS (MALDI-TOF) m/z 962.33 (M+H)⁺, calcd for C₄₉H₇₅N₁₁O₇S 962.28.

The compound of formula 5c (5 mg) was treated with TFA as described above in Example 1 for 1 h to obtain the TFA salt of 6-{2-[(4-benzothiazol-2-yl-phenyl)-methyl-amino]-ethoxy}-N-butyl-N′-[3-(1,4,7,10-tetraaza-cyclododec-1-yl)-propyl]-[1,3,5]triazine-2,4-diamine (5d). The TFA salt of 5d was used for NMR and MS characterization;

¹H NMR (MeOD): δ 7.90 (q, 4H), 7.49 (t, 1H), 7.37 (t, 1H), 6.85 (d, 2H), 4.73 (t, 2H), 3.89 (t, 2H), 3.2-3.0 (br, 12H), 2.98-2.86 (br, 4H), 2.84-2.75 (br, 4H), 2.68 (t, 3H), 1.76 (q, 2H), 1.52 (q, 2H), 1.31 (q, 2H), 0.89 (t, 3H); ¹³C NMR (CDCl₃): [symbol] 169.61, 161.13, 160.64, 160.32, 152.72, 151.72, 151.55, 133.49, 128.69, 128.64, 126.35, 124.67, 121.48, 120.93, 120.16, 118.24, 114.36, 111.84, 66.28, 49.98, 46.76, 44.31, 42.01, 41.86, 40.69, 38.95, 38.65, 37.84, 37.52, 30.51, 22.62, 19.55, 12.63; HRMS m/z 662.4073 (M+H)⁺, calcd for C₃₄H₅₂N₁₁OS 662.4077.

To the solution obtained by dissolving the TFA salt of the compound of formula 5d in methanol in a concentration of about 3 mg/50 μL, 5-7 equivalents of LiOH was added followed by an equivalent amount of CoCl₂.H₂O to prepare the Co^{II} complex of the compound of formula 5d. The complex was stirred for 1 day in the air to oxidize the Co^{II} complex to the Co^{III} complex. Oxidation of Co^{II} to Co^{III} was accompanied by appearance of deep violet color. The Co^{III} complex was isolated with HPLC by detecting at 545 nm, and evaporated to produce a solid. The solid was dissolved in water and left at room temperature for several days to obtain the stock solution of cleavage agent E. The cobalt content was measured by ICP to determine the concentration of the cleavage agent in the solution.

Activity Test of Cleavage Agent E

(1) Cleavage of Oligomers of Amylin Associated with Type 2 Diabetes Mellitus

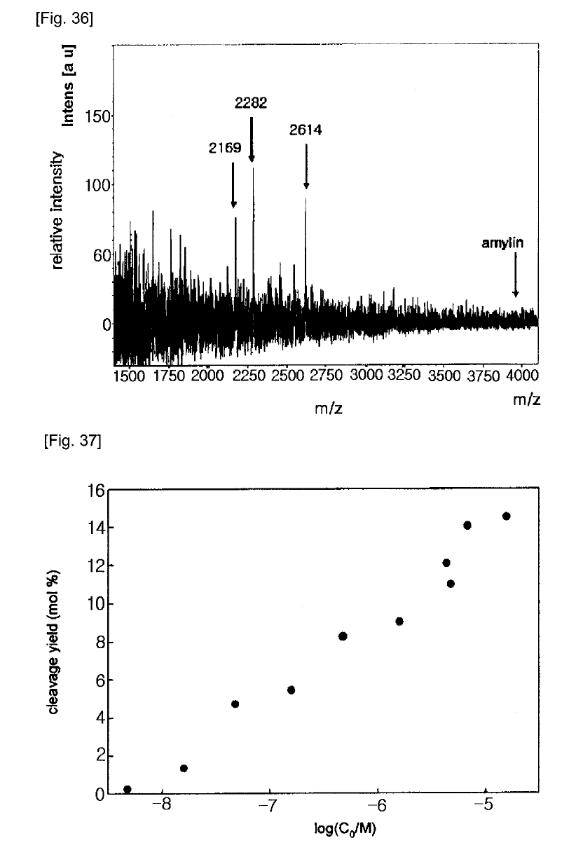

MALDI-TOF mass spectrum of the products purified with HPLC after incubating Am (4.0 μM) with cleavage agent E is illustrated in FIG. 36. As shown in FIG. 36, Am was cleaved by cleavage agent E, and Am₁₇₋₃₇, Am₁₆₋₃₇ and Am₁₃₋₃₇ were included in the cleavage products.

The cleavage yields measured after reacting Am (4.0 μM) with various concentrations of cleavage agent E at 37° C. and pH 7.50 for 36 hours are illustrated in FIG. 37.

(2) Reaction with Control Peptides or Proteins

The control experiment, identical to that of Example 1, was carried out for cleavage agent E. The results of the control experiment were the same as those obtained in Example 1.

### Example 6

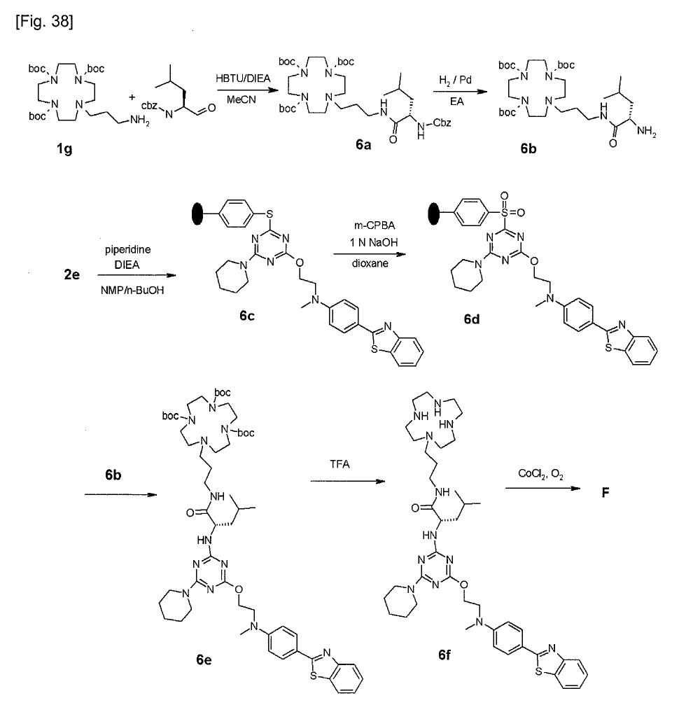

Cleavage agent F synthesized according to the pathway shown in FIG. 38.

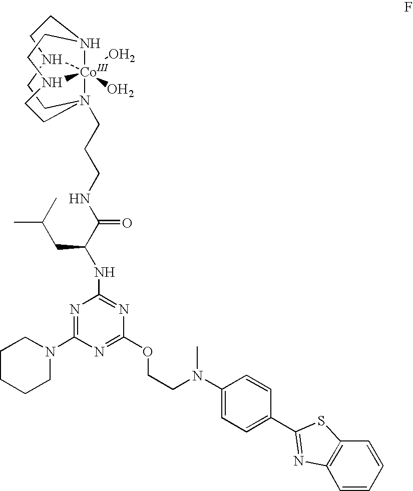

Synthesis of Compounds of Formulas 6a and 6b

To the stirred solution of the compound of formula 1g (2.9 g, 5.5 mmol) in acetonitrile (100 mL), N-α-Cbz-L-leucine (0.8 g, 5.5 mmol) and DIEA (2.9 mL, 17 mmol) were added. To the reaction mixture, HBTU (2.1 g, 5.5 mmol) was added and the mixture was stirred for 1 hour. The residue obtained by evaporation of the solution was dissolved in EA (100 mL). The EA solution was washed with 5% aq. citric acid (50 mL), 5% aq. Na₂CO₃ (50 mL), and brine (50 mL), and dried over Na₂SO₄. The solvent was evaporated off and column chromatography afforded 10-[(R)-3-(4-methyl-2-phenoxycarbonylamino-pentanoylamino)-propyl]-1,4,7,10-tetraaza-cyclododecane-1,4,7-tricarboxylic acid tri-tert-butyl ester (6a) as a colorless oil.

R_{f} 0.5 (EA/hexane 2:1); ¹H NMR (CDCl₃): δ 7.31-7.26 (br, 5H), 4.75-4.33 (br, 2H), 3.63-3.38 (br, 15H), 2.67-2.29 (br, 6H), 1.71-1.58 (m, 5H), 1.50-1.29 (br, 27H), 0.96-0.92 (t, 6H) MS (MALDI-TOF) m/z MS (MALDI-TOF) m/z 776.63 (M+H)⁺, calcd for C₄₀H₆₈N₆O₉ 776.50.

A suspension of the compound of formula 6a (1.8 g, 2.7 mmol) and 1.0 g of 10% Pd/C in 80 mL of EA was stirred under 1 atm of H₂ for 24 hours. The catalyst was filtered off on Celite, and the solvent was evaporated off to afford 10-[3-(2-amino-4-methyl-pentanoylamino)-propyl]-1,4,7,10-tetraaza-cyclododecane-1,4,7-tricarboxylic acid tri-tert-butyl ester (6b) as a solid

¹H NMR (CDCl₃): [symbol] 7.72 (s, 1H), 4.60-4.20 (br, 2H), 3.70-3.13 (br, 15H), 2.75-2.50 (br, 6H), 1.86-1.62 (m, 4H), 1.54-1.36 (m, 28H), 1.05-0.84 (t, 6H); ¹³C NMR (MeOD): δ [symbol] 176.12, 156.31, 156.11, 155.91, 79.70, 54.16, 53.91, 53.18, 49.23, 46.84, 43.98, 37.53, 37.70, 27.70, 27.52, 24.51, 22.84, 22.02, 21.26; MS (MALDI-TOF) m/z 643.57 (M+H)⁺, calcd for C₃₂H₆₂N₆O₇ 643.47.

Synthesis of Resins of Formulas 6c and 6d

To the suspension of the resin of formula 2e (55 mg, 0.15 mmol) in NMP (1 mL), n-butanol (1 mL) and piperidine (57 μL, 0.58 mmol) were added followed by DIEA (120 μL, 0.87 mmol). The mixture was heated at 120° C. for 8 hours. After filtration, the resulting resin (6c) was washed with DMF, MC, MeOH, and MC (each 3 mL×3) and dried under nitrogen gas.

To the resin of formula 6c, the mixture of a solution of m-CPBA (130 mg, 0.74 mmol) in 1,4-dioxane (1.8 mL) and aqueous 1 N NaOH (160 mL) were added. The mixture was shaken for 4 hours at room temperature. After filtration, the resulting resin (6d) was washed with 1,4-dioxane and MC (each 3 mL×3) and was dried under nitrogen gas.

Synthesis of Compounds of Formulas 6e and 6f and Cleavage Agent F

To the suspension of the resin of formula 6d in acetonitrile (1.5 mL), PS-DIEA resin (30 mg, 0.12 mmol) and the compound of formula 6b (48 mg, 0.074 mmol) were added. The mixture was heated at 80° C. for 8 hours. The mixture was filtered and the resin was washed with MC (1 mL×3). The combined filtrate and washing were evaporated in vacuo and the residue was purified by column chromatography to obtain 10-{(R)-3-[2-(4-{2-[(4-benzothiazol-2-yl-phenyl)-methyl-amino]-ethoxy}-6-piperidin-1-yl-[1,3,5]triazin-2-ylamino)-4-methyl-pentanoylamino]-propyl}-1,4,7,10-tetraaza-cyclodode-cane-1,4,7-tricarboxylic acid tri-tert-butyl ester (6e).

R_{f} 0.2 (EA/hexane 2:1); ¹H NMR (CDCl₃): [symbol] 7.96 (q, 3H), 7.84 (d, 1H), 7.45 (t, 1H), 7.30 (t, 1H), 6.78 (d, 2H), 4.56-4.55 (br, 1H), 4.45 (t, 2H), 3.78 (t, 2H), 3.69-3.67 (br, 4H), 3.61-3.21 (br, 14H), 3.11 (s, 3H), 2.75-2.31 (br, 6H), 1.72 (m, 1H), 1.72-1.50 (br, 10H), 1.49-1.29 (br, 27H), 0.93 (m, 6H); ¹³C NMR (CDCl₃): [symbol] 173.17, 170.46, 168.57, 166.78, 165.42, 156.13, 155.78, 155.30, 154.41, 150.89, 134.53, 129.00, 125.99, 124.23, 122.30, 121.60, 121.36, 111.56, 79.44, 76.61, 62.79, 54.88, 53.95, 51.10, 49.96, 49.15, 48.14, 44.46, 41.68, 39.10, 36.99, 29.69, 28.66, 28.51, 25.77, 24.87, 24.72, 24.46, 23.25, 21.80; MS (MALDI-TOF) m/z 1087.60 (M+H)⁺, calcd for C₅₆H₈₆N₁₂O₈S 1087.64.

The compound of formula 6e was treated with TFA as described above in Example 1 for 1 h to obtain the TFA salt of 2-((S)-4-{2-[(4-benzothiazol-2-yl-phenyl)-methyl-amino]-ethoxy}-6-piperidin-1-yl-[1,3,5]triazin-2-ylamino)-4-methyl-pentanoic acid [3-(1,4,7,10-tetraaza-cyclododec-1-yl)-propyl]-amide (6f). The TFA salt of the compound of formula 6f was used for NMR and MS characterization.

¹H NMR (MeOD): [symbol] 7.90 (q, 4H), 7.48 (t, 1H), 7.37 (t, 1H), 6.88 (d, 2H), 4.70-4.68 (br, 1H), 4.52 (t, 2H), 3.89 (t, 2H), 3.82-3.68 (br, 4H), 3.20-3.05 (br, 13H), 3.02-2.93 (br, 4H), 2.85-2.80 (br, 4H), 2.59 (t, 2H), 1.75-1.50 (br, 7H), 0.89 (t, 6H); ¹³C NMR (MeOD): δ 173.16, 169.35, 161.64, 161.45, 158.40, 157.85, 157.30, 153.47, 151.32, 133.84, 128.59, 126.14, 124.46, 121.35, 120.62, 116.53, 111.61, 65.65, 65.47, 53.59, 50.18, 50.01, 46.77, 45.25, 44.06, 42.06, 41.84, 41.09, 37.83, 36.60, 25.09, 24.54, 23.81, 21.90, 20.64, 14.04; HRMS m/z 787.4913 (M+H)⁺, calcd for C₄₁H₆₃N₁₂O₂S 787.4918.

The stock solution of cleavage agent F was obtained from the compound of formula 6f as described for cleavage agent E in Example 5.

Activity Test of Cleavage Agent F

(1) Cleavage of Oligomers of Amylin Associated with Type 2 Diabetes Mellitus

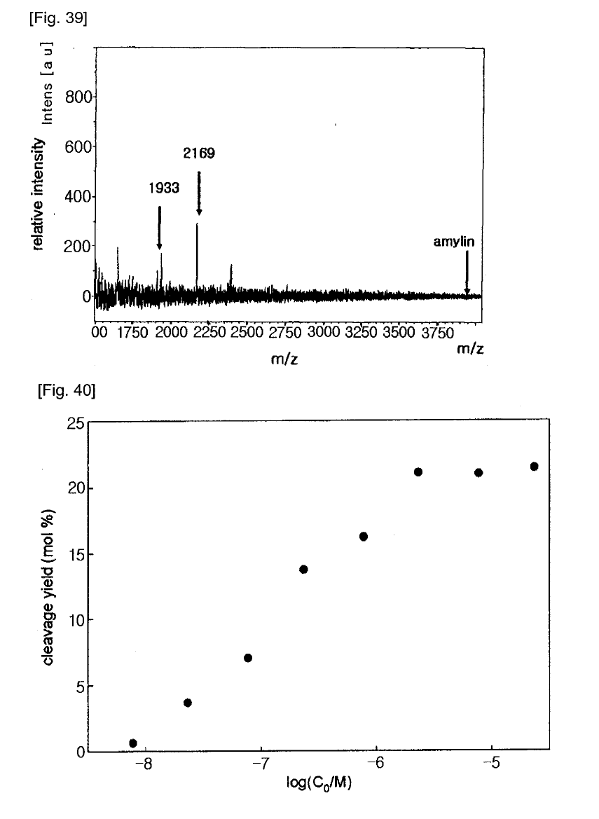

MALDI-TOF mass spectrum of the products purified with HPLC after incubating Am (4.0 μM) with cleavage agent F is illustrated in FIG. 39. As shown in FIG. 39, Am was cleaved by cleavage agent F, and Am₁₉₋₃₇ and Am₁₇₋₃₇ were included in the cleavage products.

The cleavage yields measured after reacting Am (4.0 μM) with various concentrations of cleavage agent F at 37° C. and pH 7.50 for 36 hours are illustrated in FIG. 40.

(2) Reaction with Control Peptides or Proteins

The control experiment, identical to that of Example 1, was carried out for cleavage agent F. The results of the control experiment were the same as those obtained in Example 1.

### Example 7

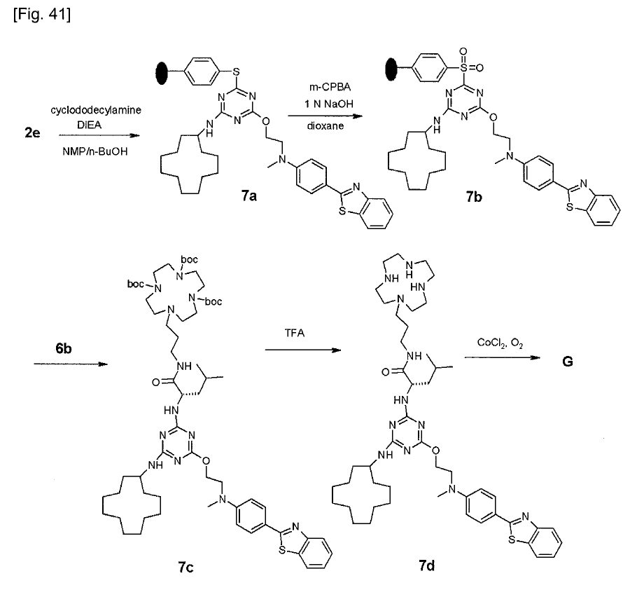

Cleavage agent G was synthesized according to the pathway shown in FIG. 41.

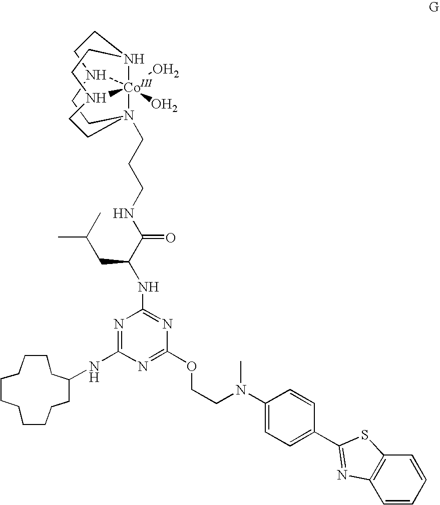

Synthesis of Resins of Formulas 7a and 7b

To the suspension of 2e (55 mg, 0.15 mmol) in NMP (1 mL), n-butanol (1 mL) and cyclododecylamine (75 μL, 0.58 mmol), followed by DIEA (120 μL, 0.87 mmol) were added. The mixture was heated at 120° C. for 8 hours. After filtration, the resulting resin (7a) was washed with DMF, MC, MeOH, and MC (each 3 mL×3) and dried under nitrogen gas.

To the resin of formula 7a was added the mixture of a solution of m-CPBA (130 mg, 0.74 mmol) in 1,4-dioxane (1.8 mL) and aqueous 1 N NaOH (160 μL). The mixture was shaken for 4 hours at room temperature. After filtration, the resulting resin (7b) was washed with 1,4-dioxane and MC (each 3 mL×3) and was dried under nitrogen gas.

Synthesis of Compounds of Formulas 7c and 7d and Cleavage Agent F

To the suspension of the resin of formula 7b in acetonitrile (1.5 mL) were added PS-DIEA resin (30 mg, 0.12 mmol) and the compound of formula 6b (48 mg, 0.074 mmol). The mixture was heated at 80° C. for 8 hours. The mixture was filtered and the resin was washed with MC (1 mL×3). The combined filtrate and washing were evaporated in vacuo and the residue was purified by column chromatography to obtain 10-{(R)-3-[2-(4-{2-[(4-benzothiazol-2-yl-phenyl)-methyl-amino]-ethoxy}-6-cyclododecylamino-[1,3,5]triazin-2-ylamino)-4-methyl-pentanoylamino]-propyl}-1,4,7,10-tetraaza-cyclododecane-1,4,7-tricarboxylic acid tri-tert-butyl ester (7c).

R_{f} 0.2 (EA/hexane 2:1); ¹H NMR (CDCl₃): [symbol] 7.96 (q, 3H), 7.84 (d, 1H), 7.43 (t, 1H), 7.30 (t, 1H), 6.77 (d, 2H), 4.47-4.41 (br, 3H), 4.14-4.04 (br, 1H), 3.77 (t, 2H), 3.68-21 (br, 14H), 3.12 (s, 3H), 2.75-2.34 (br, 6H), 1.72-1.55 (br, 5H), 1.52-1.40 (br, 27H), 1.38-1.28 (br, 22H), 0.87 (m, 6H); ¹³C NMR (CDCl₃): δ 173.56, 170.42, 168.50, 166.90, 165.93, 156.07, 155.74, 155.26, 154.39, 150.91, 134.50, 128.97, 125.95, 124.20, 122.26, 121.62, 121.53, 121.32, 111.59, 111.49, 79.49, 79.34, 79.23, 76.73, 63.17, 63.06, 51.05, 49.87, 47.99, 47.55, 47.32, 39.21, 38.83, 30.58, 29.65, 28.64, 28.49, 25.00, 24.83, 24.10, 23.95, 23.73, 23.51, 23.32, 23.08, 22.15, 21.74, 21.17; MS (MALDI-TOF) m/z 1185.53 (M+H)⁺, calcd for C₆₃H₁₀₀N₁₂O₈S 1185.75.

The compound of formula 7c was treated with TFA as described above in Example 1 for 1 h to obtain the TFA salt of 2-((S)-4-{2-[(4-benzothiazol-2-yl-phenyl)-methyl-amino]-ethoxy}-6-cyclododecylamino-[1,3,5]triazine-2-ylamino)-4-methyl-pentanoic acid [3-(1,4,7,10-tetraaza-cyclodode-1-sil)-propyl]-amide (7d). The TFA salt of the compound of formula 7d was used for NMR and MS characterization.

¹H NMR (MeOD): [symbol] 7.90 (q, 4H), 7.47 (t, 1H), 7.35 (t, 1H), 6.81 (d, 2H), 4.80-4.53 (br, 3H), 4.12-4.03 (br, 1H), 3.89 (t, 2H), 3.22-3.12 (br, 10H), 3.10-3.00 (br, 3H), 2.97-2.92 (br, 4H), 2.90-2.73 (br, 4H), 2.63 (t, 2H), 1.76-1.60 (br, 5H), 1.40-1.12 (br, 22H), 0.72 (t, 6H); ¹³C NMR (MeOD): [symbol] 172.79, 169.50, 161.97, 161.41, 160.99, 159.38, 157.83, 157.28, 151.60, 133.48, 128.80, 126.32, 124.63, 121.45, 120.93, 116.52, 111.63, 66.45, 65.47, 53.76, 49.94, 46.79, 44.15, 41.96, 41.92, 37.52, 24.66, 23.61, 23.53, 23.37, 23.52, 23.10, 22.90, 22.10, 20.86, 20.46, 14.06; HRMS m/z 885.5991 (M+H)⁺, calcd for C₄₈H₇₇N₁₂O₂S 885.6013.

The stock solution of cleavage agent G was obtained from the compound of formula 7d as described for cleavage agent E in Example 5.

Activity Test of Cleavage Agent G

(1) Cleavage of Oligomers of Amylin Associated with Type 2 Diabetes Mellitus

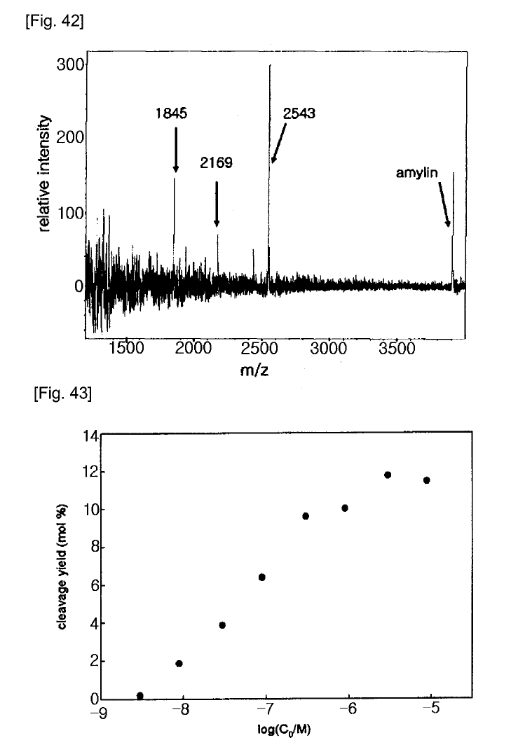

MALDI-TOF mass spectrum of the products purified with HPLC after incubating Am (4.0 μM) with cleavage agent G is illustrated in FIG. 42. As shown in FIG. 42, Am was cleaved by cleavage agent F, and Am₂₀₋₃₇, Am₁₇₋₃₇ and Am₁₄₋₃₇ were included in the cleavage products.

The cleavage yields measured after reacting Am (4.0 μM) with various concentrations of cleavage agent G at 37° C. and pH 7.50 for 36 hours are illustrated in FIG. 43.

(2) Reaction with Control Peptides or Proteins

The control experiment, identical to that of Example 1, was carried out for cleavage agent G. The results of the control experiment were the same as those obtained in Example 1.

### Example 8

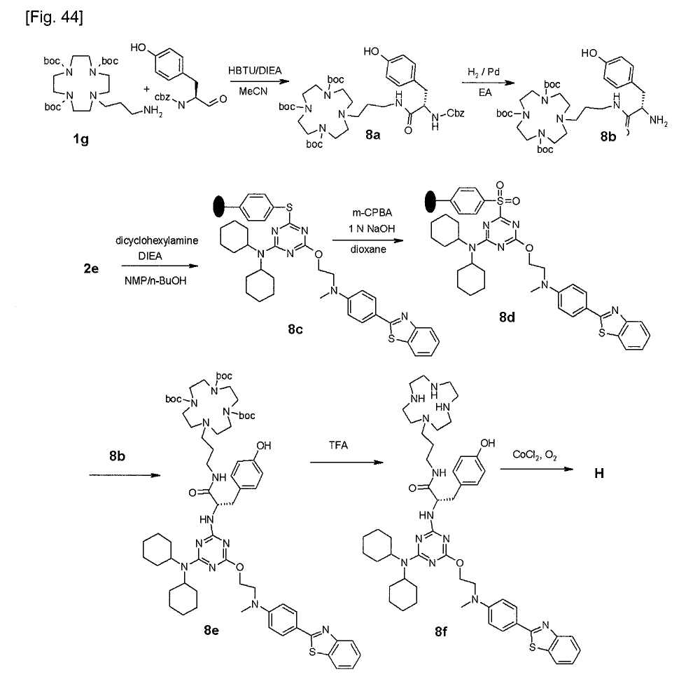

Cleavage agent H was synthesized according to the pathway shown in FIG. 44.

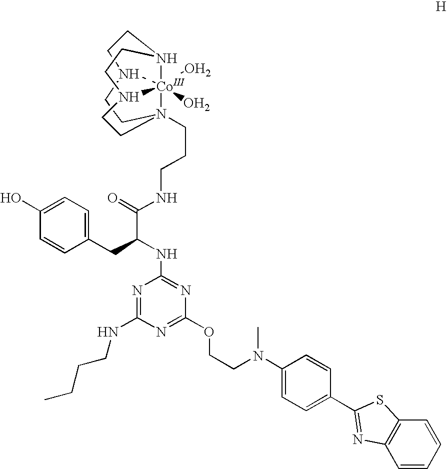

Synthesis of Compounds of Formulas 8a and 8b

To the stirred solution of the compound of formula 1g (2.9 g, 5.5 mmol) in acetonitrile (100 mL), N-α-Cbz-L-tyrosine (1.1 g, 5.5 mmol) and DIEA (2.9 mL, 17 mmol) were added. To the reaction mixture was added HBTU (2.1 g, 5.5 mmol) and the mixture was stirred for 1 hour. The residue obtained by evaporation of the solution was dissolved in EA (100 mL). The EA solution was washed with 5% aq. citric acid (50 mL), 5% aq. Na₂CO₃ (50 mL), and brine (50 mL), and dried over Na₂SO₄. The solvent was evaporated off and column chromatography afforded 10-{(S)-3-[3-(4-hydroxy-phenyl)-2-phenoxycarbonylamino-propionylamino]-propyl}-1,4,7,10-tetraaza-cyclododecane-1,4,7-tricarboxylic acid tri-tert-butyl ester (8a) as a colorless oil.

R_{f} 0.5 (EA/hexane 3:1). ¹H NMR (CDCl₃): [symbol] 7.42-7.23 (br, 5H), 7.07-6.85 (d, 2H), 6.77-6.62 (d, 2H), 4.60-4.13 (br, 4H), 3.77-2.92 (br, 16H), 2.69-2.54 (br, 4H), 2.50-2.37 (br, 2H), 1.65-1.31 (br, 29H); MS (MALDI-TOF) m/z 827.75 (M+H)⁺, calcd for C₄₃H₆₆N₆O₁₀ 827.04.

A suspension of the compound of formula 8a (2.0 g, 2.7 mmol) and 1.0 g of 10% Pd/C in 80 mL of EA was stirred under 1 atm of H₂ for 24 hours. The catalyst was filtered off on Celite, and the solvent was evaporated off to afford 10-{(R)-3-[2-amino-3-(4-hydroxy-phenyl)-propionylamino]-propyl}-1,4,7,10-tetraaza-cyclododecane-1,4,7-tricarboxylic acid tri-tert-butyl ester (8b) as a solid.

¹H NMR (CDCl₃): [symbol] 7.2 (s, 11H), 7.05-6.97 (d, 2H), 6.81-6.72 (d, 2H), 3.65-3.58 (m, 1H), 3.53-3.20 (br, 14H), 3.12-3.00 (m, 2H), 2.75-2.48 (br, 8H), 1.76-1.64 (m, 2H), 1.54-1.38 (m, 27H); ¹³C NMR (CDCl₃): [symbol] 173.51, 155.91, 155.51, 130.40, 127.44, 115.62, 79.91, 79.65, 76.58, 56.12, 54.55, 49.76, 47.73, 39.63, 38.56, 37.13, 29.62, 28.61, 28.45, 24.23; MS (MALDI-TOF) m/z 692.84 (M+H)⁺, calcd for C₃₅H₆₀N₆O₈ 692.90.

Synthesis of Resins of Formulas 8c and 8d

To the suspension of the resin of formula 2e (55 mg, 0.15 mmol) in NMP (1 mL), n-butanol (1 mL) and dicyclohexylamine (115 μL, 0.58 mmol) were added followed by DIEA (120 μL, 0.87 mmol). The mixture was heated at 120° C. for 8 hours. After filtration, the resulting resin (8c) was washed with DMF, MC, MeOH, and MC (each 3 mL×3) and dried under nitrogen gas.

To the resin of formula 8c, the mixture of a solution of m-CPBA (130 mg, 0.74 mmol) in 1,4-dioxane (1.8 mL) and aqueous 1 N NaOH (160 μL) were added. The mixture was shaken for 4 hours at room temperature. After filtration, the resulting resin (8d) was washed with 1,4-dioxane and MC (each 3 mL×3) and was dried under nitrogen gas.

Synthesis of Compounds of Formulas 8e and 8f and Cleavage Agent H

To the suspension of 8d in acetonitrile (1.5 mL), PS-DIEA resin (30 mg, 0.12 mmol) and the compound of formula 8b (51 mg, 0.074 mmol) were added. The reaction mixture was heated at 80° C. for 8 hours. The mixture was filtered and the resin was washed with MC (1 mL×3). The combined filtrate and washing were evaporated in vacuo and the residue was purified by column chromatography to obtain 10-{(S)-3-[2-(4-{2-[(4-benzothiazol-2-yl-phenyl)-methyl-amino]-ethoxy}-6-dicyclohexylamino-[1,3,5]triazin-2-ylamino)-3-(4-hydroxy-phenyl)-propionylamino]-propyl}-1,4,7,10-tetra-aza-cyclododecane-1,4,7-tricarboxylic acid tri-tert-butyl ester (8e).

R_{f} 0.2 (EA:hexane 1:4); ¹H NMR (CDCl₃): [symbol] 7.87 (t, 3H), 7.73 (d, 2H), 7.35 (t, 2H), 7.20 (m, 2H), 6.75-6.48 (br, 4H), 4.45-4.41 (m, 3H), 3.73-3.68 (m, 2H), 3.62-3.08 (br, 18H), 3.05-2.65 (br, 6H), 2.19-2.17 (m, 2H), 1.93-1.85 (m, 2H), 1.75-1.62 (br, 4H), 1.60-1.42 (br, 31H), 1.58-1.20 (br, 12H); ¹³C NMR (CDCl₃): [symbol] 169.55, 168.64, 165.09, 154.35, 151.53, 150.73, 135.81, 134.52, 129.04, 128.26, 125.53, 124.28, 122.30, 121.75, 121.38, 111.68, 76.71, 64.43, 64.24, 56.28, 56.16, 50.96, 47.21, 39.29, 34.24, 30.35, 29.99, 29.80, 29.72, 28.58, 28.46, 26.12, 25.58, 25.37, 21.22, 20.25, 20.18, 20.09; HRMS m/z 1232.7149 (M+H)⁺, calcd for C₆₆H₉₆N₁₂O₉S 1232.7144.

The compound of formula 8c was treated with TFA as described above in Example 1 for 1 h to obtain the TFA salt of 2-(4-{(S)-2-[(4-benzothiazol-2-yl-phenyl)-methyl-amino]-ethoxy}-6-dicyclohexylamino-[1,3,5]triazine-2-ylamino)-3-(4-hydroxy-phenyl)-N-[3-(1,4,7,10-tetraaza-cyclodode-1-sil)-propyl]-propionamide (8f). The TFA salt of the compound of formula 8f was used for NMR and MS characterization

¹H NMR (CDCl₃): δ 8.04 (t, 3H), 7.75 (d, 2H), 7.53 (t, 2H), 7.42 (m, 2H), 6.78-6.64 (br, 4H), 4.48-4.45 (m, 3H), 3.81-3.74 (m, 2H), 3.63-2.75 (br, 24H), 2.25-2.15 (m, 3H), 1.85-1.63 (br, 12H), 1.60-1.42 (br, 10H); ¹³C NMR (CDCl₃): [symbol] δ 169.55, 168.64, 164.59, 160.23, 153.72, 142.86, 130.91, 128.97, 128.26, 125.52, 122.10, 118.18, 117.51, 113.69, 79.80, 74.60, 73.43, 56.70, 56.53, 39.59, 34.22, 30.88, 30.47, 30.31, 29.56, 27.98, 25.25, 24.93, 21.19, 19.92; HRMS m/z 932.5573 (M+H)⁺, calcd for C₅₁H₇₂N₁₂O₃S 932.5571.

The stock solution of cleavage agent H was obtained from the compound of formula 8f as described for cleavage agent E in Example 5.

Activity Test of Cleavage Agent H

(1) Cleavage of Oligomers of Amylin Associated with Type 2 Diabetes Mellitus

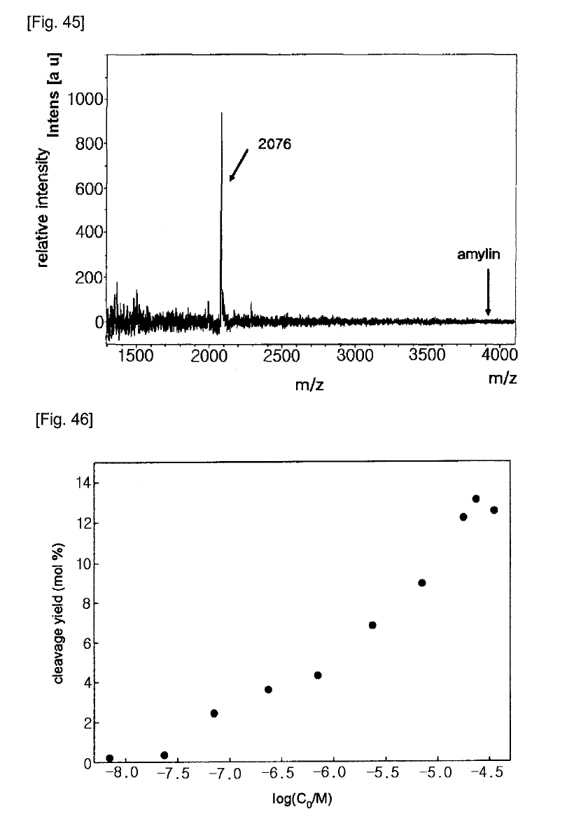

MALDI-TOF mass spectrum of the products purified with HPLC after incubating Am (4.0 μM) with cleavage agent H is illustrated in FIG. 45. As shown in FIG. 45, Am was cleaved by cleavage agent H, and Am₁₋₁₉ was included in the cleavage products.

The cleavage yields measured after reacting Am (4.0 μM) with various concentrations of cleavage agent H at 37° C. and pH 7.50 for 36 hours are illustrated in FIG. 46.

(2) Reaction with Control Peptides or Proteins

The control experiment, identical to that of Example 1, was carried out for cleavage agent H. The results of the control experiment were the same as those obtained in Example 1.

1. A cleavage agent of formula 1 selectively acting on soluble assembly of amyloidogenic peptide or protein:
(R)ₙ-(L)ₘ-Z [formula 1]
wherein,
R is a target recognition site independently selected from the group consisting of A, A-(Y)ₒ—(CH₂)ₚ—(Y)ₒ-A, A-(CH═CH)-A, A-(Y)ₒ—(CH₂)ₚ—(Y)ₒ-A-(Y)ₒ—(CH₂)ₚ—(Y)ₒ-A and A-(Y)ₒ—(CH₂)ₚ—(Y)ₒ-A-(Y)ₒ—(CH₂)ₚ—(Y)ₒ-A-(Y)ₒ-(CH₂)ₚ—(Y)ₒ-A,
A is independently C₆₋₁₄aryl, or 5- to 14-membered heteroaryl having one or more hetero atom(s) selected from the group consisting of oxygen, sulfur and nitrogen,
wherein, aryl or heteroaryl is unsubstituted or substituted with one or more substituent(s) independently selected from the group consisting of C₁₋₁₅alkyl, hydroxy, C₁₋₁₅alkoxy, C₁₋₁₅alkylcarbonyloxy, C₁₋₁₅alkylsulfonyloxy, amino, mono or diC₁₋₁₅alkylamino, C₁₋₁₅alkylcarbonylamino, C₁₋₁₅alkylsulfonylamino, C₃₋₁₅cycloalkylamino, formyl, C₁₋₁₅alkylcarbonyl, carboxy, C₁₋₁₅alkyloxycarbonyl, carbamoyl, mono or diC₁₋₁₅alkylcarbamoyl, C₁₋₁₅alkylsulfanylcarbonyl, C₁₋₁₅alkylsulfanylthiocarbonyl, C₁₋₁₅alkoxycarbonyloxy, carbamoyloxy, mono or diC₁₋₁₅alkylcarbamoyloxy, C₁₋₁₅alkylsulfanylcarbonyloxy, C₁₋₁₅alkoxycarbonylamino, ureido, mono or di or triC₁₋₁₅alkylureido, C₁₋₁₅alkylsulfanylcarbonylamino, mercapto, C₁₋₁₅alkylsulfanyl, C₁₋₁₅alkyldisulfanyl, sulfo, C₁₋₁₅alkoxysulfonyl, sulfamoyl, mono or diC₁₋₁₅alkylsulfamoyl, triC₁₋₁₅alkylsilanyl and halogen;
Y is O or N-Z, wherein Z is hydrogen or C₁₋₉alkyl;
L is a linker;
Z is a metal ion-ligand complex as a catalytic site;
n is an independent integer from 1 to 6;
m and o are independently 0 or 1;
p is an integer from 0 to 5.

2. The cleavage agent of claim 1, wherein A is selected from the group consisting of the following formulas; and p is independently 0, 1 or 2: 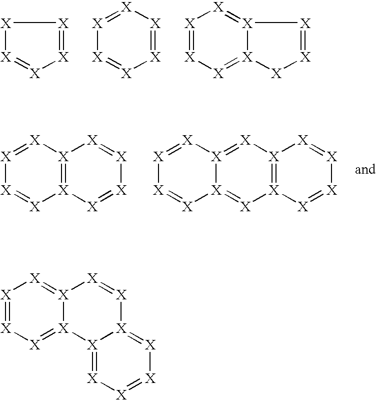
wherein,
X is independently selected from the group consisting of C, N, NH, O and S,
A is unsubstituted or substituted with one or more substituent(s) independently selected from the group consisting of C₁₋₄alkyl, C₁₋₄alkoxy, amino, mono or diC₁₋₁₂alkylamino, C₁₋₆alkylcarbonylamino, C₁₋₆alkylsulfonylamino, C₅₋₁₅cycloalkylamino, C₁₋₆alkylcarbonyl, carbamoyl, mono or diC₁₋₆alkylcarbamoyl, C₁₋₆alkoxycarbonylamino, ureido, mono or di or triC₁₋₆alkylureido, C₁₋₆alkylsulfanylcarbonylamino, C₁₋₆alkylsulfanyl, C₁₋₆alkyldisulfanyl, sulfamoyl, mono or diC₁₋₆alkylsulfamoyl, triC₁₋₁₅alkylsilanyl and halogen.

3. The cleavage agent of claim 2, wherein A is selected from the group consisting of the following formulas: 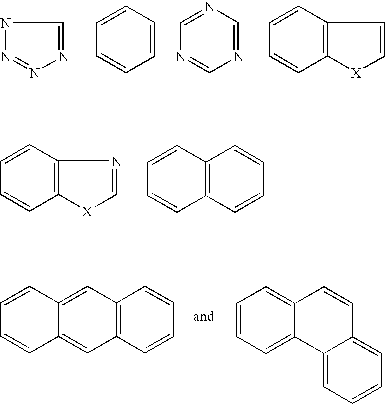
wherein,
X is NH, O, or S,
A is unsubstituted or substituted with one or more substituent(s) independently selected from the group consisting of C₁₋₄alkyl, C₁₋₄alkoxy, amino, mono or diC₁₋₈alkylamino, C₆₋₁₂cycloalkylamino, C₁₋₄alkylcarbonyl and halogen.

4. The cleavage agent of claim 1, wherein the ligand is selected from the group consisting of the following formulas: 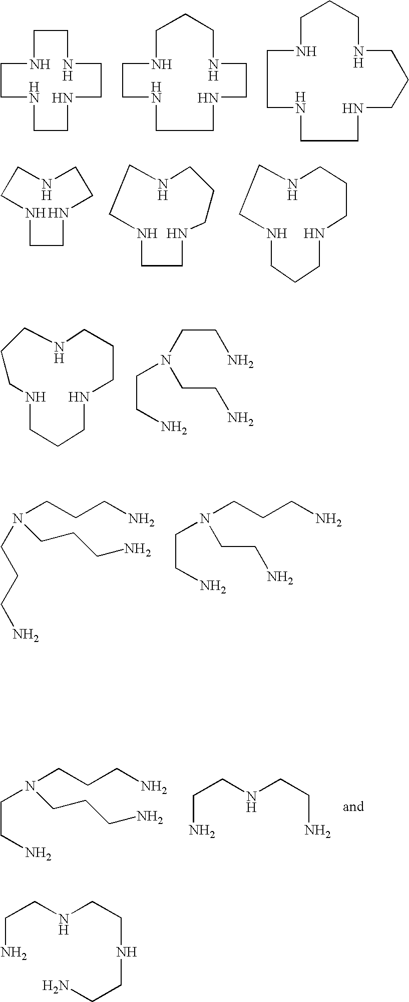
wherein,
the nitrogen atom in the ligand may be replaced with an atom selected from the group consisting of oxygen, sulfur and phosphorous;
the ligand may be fused with C₆₋₁₄aryl or 5- to 14-membered heteroaryl;
the ligand is unsubstituted or substituted with one or more substituent(s) independently selected from the group consisting of C₁₋₁₅alkyl, hydroxy, C₁₋₁₅alkoxy, C₁₋₁₅alkylcarbonyloxy, C₁₋₁₅alkylsulfonyloxy, amino, mono or diC₁₋₁₅alkylamino, C₁₋₁₅alkylcarbonylamino, C₁₋₁₅alkylsulfonylamino, formyl, C₁₋₁₅alkylcarbonyl, carboxy, C₁₋₁₅alkyloxycarbonyl, carbamoyl, mono or diC₁₋₁₅alkylcarbamoyl, C₁₋₁₅alkylsulfanylcarbonyl, C₁₋₁₅alkylsulfanylthiocarbonyl, C₁₋₁₅alkoxycarbonyloxy, carbamoyloxy, mono or diC₁₋₁₅alkylcarbamoyloxy, C₁₋₁₅alkylsulfanylcarbonyloxy, C₁₋₁₅alkoxycarbonylamino, ureido, mono or di or triC₁₋₁₅alkylureido, C₁₋₁₅alkylsulfanylcarbonylamino, mercapto, C₁₋₁₅alkylsulfanyl, C₁₋₁₅alkyldisulfanyl, sulfo, C₁₋₅alkoxysulfonyl, sulfamoyl, mono or diC₁₋₁₅alkylsulfamoyl, triC₁₋₁₅alkylsilanyl and halogen.

5. The cleavage agent of claim 1, wherein the ligand is selected from the group consisting of the following formulas: 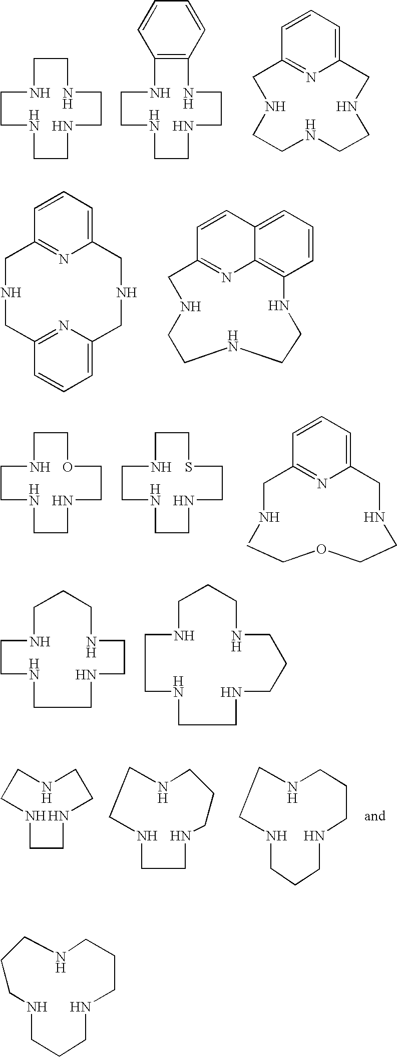
wherein,
the ligand is unsubstituted or substituted with one or more substituent(s) independently selected from the group consisting of C₁₋₆alkyl, C₁₋₆alkoxy, C₁₋₆alkylcarbonyloxy and halogen.

6. The cleavage agent of claim 1, wherein the metal ion is selected from the group consisting of Co^{III}, Cu^{I}, Cu^{II}, Ce^{IV}, Ce^{V}, Cr^{III}, Fe^{II}, Fe^{III}, Mo^{IV}, Ni^{II}, Pd^{II}, Pt^{II}, V^{V} and Zr^{IV}.

7. The cleavage agent of claim 6, wherein the metal ion is Co^{III}, Cu^{II} or Pd^{II}.

8. The cleavage agent of claim 1, wherein the linker (L) is comprised of a backbone comprising 1 to 30 atom(s) independently selected from the group consisting of carbon, nitrogen, oxygen, silicon, sulfur and phosphorous,
wherein, the atom in the backbone is present as the form of a functional group independently selected from the group consisting of alkane, alkene, alkyne, carbonyl, thiocarbonyl, amine, ether, silyl, sulfide, disulfide, sulfonyl, sulfinyl, phosphoryl, phosphinyl, amide, imide, ester and thioester, and
wherein the linker is unsubstituted or substituted with one or more substituent(s) independently selected from the group consisting of C₁₋₉alkyl, hydroxy, C₁₋₉alkoxy, C₁₋₉alkylcarbonyloxy, C₁₋₉alkylsulfonyloxy, amino, mono or diC₁₋₉alkylamino, C₁₋₉alkylcarbonylamino, C₁₋₉alkylsulfonylamino, formyl, C₁₋₉alkylcarbonyl, carboxy, C₁₋₉alkyloxycarbonyl, carbamoyl, mono or diC₁₋₉alkylcarbamoyl, C₁₋₉alkylsulfanylcarbonyl, C₁₋₉alkylsulfanylthiocarbonyl, C₁₋₉alkoxycarbonyloxy, carbamoyloxy, mono or diC₁₋₉alkylcarbamoyloxy, C₁₋₉alkylsulfanylcarbonyloxy, C₁₋₉alkoxycarbonylamino, ureido, mono or di or triC₁₋₉alkylureido, C₁₋₉alkylsulfanylcarbonylamino, mercapto, C₁₋₉alkylsulfanyl, C₁₋₉alkyldisulfanyl, sulfo, C₁₋₉alkoxysulfonyl, sulfamoyl, mono or diC₁₋₉alkylsulfamoyl, triC₁₋₉alkylsilanyl and halogen.

9. The cleavage agent of claim 8, wherein the number of atoms in the backbone is 1 to 20, and
wherein the linker is unsubstituted or substituted with one or more substituent(s) independently selected from the group consisting of C₁₋₆alkyl, C₁₋₆alkoxy, mono or diC₁₋₆alkylamino, C₁₋₆alkylcarbonylamino, C₁₋₆alkylsulfonylamino, C₁₋₆alkylcarbonyl, carbamoyl, mono or diC₁₋₆alkylcarbamoyl, C₁₋₆alkoxycarbonylamino, ureido, mono or di or triC₁₋₆alkylureido, C₁₋₆alkylsulfanylcarbonylamino, C₁₋₆alkylsulfanyl, C₁₋₆alkyldisulfanyl, sulfamoyl, mono or diC₁₋₆alkylsulfamoyl, triC₁₋₆alkylsilanyl and halogen.

10. The cleavage agent of claim 1, wherein one or more of R, L and Z of the compound of formula 1 is further substituted with -(L)ₘ-(R)ₙ, wherein R, Z, L, m and n are the same as defined in claim 1.

11. The cleavage agent of claim 1, wherein the agent cleaves oligomer of Aβ₄₀ or Aβ₄₂.

12. The cleavage agent of claim 1, wherein the agent cleaves oligomer of amylin.

13. The cleavage agent of claim 1, wherein the agent cleaves oligomer of α-synuclein.

14. A pharmaceutical composition for prevention or treatment of amyloidosis, comprising the cleavage agent defined in claim 1 and pharmaceutically acceptable salts.

15. The pharmaceutical composition of claim 14, wherein the amyloidosis is Alzheimer's disease, type 2 diabetes mellitus, Parkinson's disease, spongiform encepahlopathies or Huntington's disease.
